(19) United States
(12) Patent Application Publication
(10) Pub. No.: US 2006/0069080 A1
(43) Pub. Date: Mar. 30, 2006

(54) Combinations of substituted azetidinones and CB1 antagonists

(76) Inventor: Enrico P. Veltri, Princeton, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/236,225

(22) Filed: Sep. 27, 2005

Related U.S. Application Data
(60) Provisional application No. 60/614,167, filed on Sep. 29, 2004.

(51) Int. Cl. A61K 31/397

(52) U.S. Cl. 514/210.02

(57) ABSTRACT

The present invention provides compositions, therapeutic combinations and methods including: (a) at least one selective CB₁ antagonist; and (b) at least one substituted azetidinone or substituted β-lactam sterol absorption inhibitor which can be useful for treating vascular conditions, diabetes, obesity, metabolic syndrome and lowering plasma levels of sterols or 5α-stanols.

This application claims the benefit of U.S. Provisional Application No. 60/614,167, filed Sep. 29, 2004.

### FIELD OF THE INVENTION

The present invention relates to compositions and therapeutic combinations comprising a cholesterol lowering compound, for example a substituted azetidinone or a substituted β-lactam, and a selective cannabinoid-1 (i.e., “CB₁”) receptor antagonist for treating vascular and lipidemic conditions such as are associated with atherosclerosis, hypercholesterolemia and other vascular conditions in subjects.

### BACKGROUND OF THE INVENTION

Atherosclerotic coronary heart disease (CHD) represents the major cause for death and vascular morbidity in the western world. Risk factors for atherosclerotic coronary heart disease include hypertension, diabetes mellitus, family history, male gender, cigarette smoke and high serum cholesterol. A total cholesterol level in excess of 225-250 mg/dL is associated with significant elevation of risk of CHD. The newly revised NCEP ATP III low density lipoprotein (LDL-C) goal for patients with CHD or CHD risk equivalent is <100 mg/dL (2.59 mmol/L), for individuals with two or more risk factors is <130 mg/dL (3.37 mmol/L) and for individuals with fewer than two risk factors is <160 mg/dL (4.14 mmol/L).

The regulation of whole-body cholesterol homeostasis in mammals and animals involves the regulation of dietary cholesterol and modulation of cholesterol biosynthesis, bile acid biosynthesis and the catabolism of the cholesterol-containing plasma lipoproteins. The liver is the major organ responsible for cholesterol biosynthesis and catabolism and, for this reason, it is a prime determinant of plasma cholesterol levels. The liver is the site of synthesis and secretion of very low density lipoproteins (VLDL) which are subsequently metabolized to low density lipoproteins (LDL) in the circulation. LDL are the predominant cholesterol-carrying lipoproteins in the plasma and an increase in their concentration is correlated with increased atherosclerosis. When intestinal cholesterol absorption is reduced, by whatever means, less cholesterol is delivered to the liver. The consequence of this action is decreased hepatic lipoprotein (VLDL) production and an increase in the hepatic clearance of plasma cholesterol, mostly as LDL. Thus, the net effect of inhibiting intestinal cholesterol absorption is a decrease in plasma cholesterol levels and a decrease in the progression of atherosclerotic lesion formation.

Compounds which lower cholesterol include HMG CoA reductase inhibitor compounds, HMG CoA synthetase inhibitors, squalene synthesis inhibitors, squalene epoxidase inhibitors, sterol biosynthesis inhibitors, nicotinic acid derivatives, bile acid sequestrants, inorganic cholesterol sequestrants, AcylCoA:Cholesterol O-acyltransferaseinhibitors, cholesteryl ester transfer protein inhibitors, fish oils containing Omega 3 fatty acids, natural water soluble fibers, plant stanols and/or fatty acid esters of plant stanols, and low-density lipoprotein receptor activators.

Particularly useful cholesterol lowering compounds include hydroxy-substituted azetidinone compounds and substituted β-lactam compounds, for example those disclosed in U.S. Pat. Nos. 5,767,115, 5,624,920, 5,668,990, 5,656,624 and 5,688,787. These patents, respectively, disclose hydroxy-substituted azetidinone compounds and substituted β-lactam compounds useful for lowering cholesterol and/or in inhibiting the formation of cholesterol-containing lesions in mammalian arterial walls. U.S. Pat. No. 5,756,470, U.S. Patent Application No. 2002/0137690, U.S. Patent Application No. 2002/0137689 and PCT Patent Application No. WO 2002/066464 disclose sugar-substituted azetidinones and amino acid substituted azetidinones useful for preventing or treating atherosclerosis and reducing plasma cholesterol levels.

U.S. Pat. Nos. 5,846,966 and 5,661,145, respectively, disclose treatments for inhibiting atherosclerosis and reducing plasma cholesterol levels using such hydroxy-substituted azetidinone compounds or substituted β-lactam compounds in combination with HMG CoA reductase inhibitor compounds, which act by blocking hydroxymethylglutaryl coenzyme A (HMG-CoA) reductase (the rate-limiting enzyme in hepatic cholesterol synthesis). HMG-CoA reductase inhibitors, e.g., statins such as lovastatin, simvastatin, and pravastatin, slow the progression of atherosclerotic lesions in the coronary and carotid arteries. Simvastatin and pravastatin have also been shown to reduce the risk of coronary heart disease events in patients with hypercholesterolemia and/or CHD.

Simvastatin is marketed worldwide, and sold in the U.S. under the tradename ZOCOR®. Methods for making it are described in U.S. Pat. Nos. 4,444,784; 4,916,239; 4,820,850; among other patent and literature publications.

The CB₁ receptor is one of the most abundant neuromodulatory receptors in the brain, and is expressed at high levels in the hippocampus, cortex, cerebellum, and basal ganglia (e.g., Wilson et al., Science, 2002, vol. 296, 678-682). Selective CB₁ receptor antagonists, for example pyrazole derivatives such as rimonabant, can be used to treat various conditions, such as obesity and metabolic syndrome (e.g., Bensaid et al., Molecular Pharmacology, 2003 vol. 63, no. 4, pp. 908-914; Trillou et al., Am. J. Physiol. Regul. Integr. Comp. Physiol. 2002 vol. 284, R345-R353; Kirkham, Am. J. Physiol. Regul. Integr. Comp. Physiol. 2002 vol. 284, R343-R344; Sanofi-Aventis Publication, Bear Stearns Conference, New York, Sep. 14, 2004; Nicole Cranois and Jean-Marc Podvin, Sanofi-Synthelabo, press release reporting results of RIO-LIPIDS AND STRATUS-US Study results, American College of Cardiology Annual Meeting, New Orleans, Mar. 9, 2004), neuroinflammatory disorders (e.g., Adam, et al., Expert Opin. Ther. Patents, 2002, vol. 12, no. 10, 1475-1489), cognitive disorders, psychosis, addiction, gastrointestinal disorders (e.g., Lange et al., J. Med. Chem. 2004, vol. 47, 627-643) and cardiovascular conditions (e.g., Porter et al., Pharmacology and Therapeutics, 2001 vol. 90, 45-60).

Recently, it has been shown that treatments of subjects with CB₁ receptor antagonists (e.g., rimonabant) can increase serum high density lipoprotein (HDL) levels and decrease triglyceride levels in patients (Sanofi-Aventis Publication, Bear Stearns Conference, New York, Sep. 14, 2004, pages 19-24).

Despite recent improvements in the treatment of vascular disease, there remains a need for improved compounds, compositions and treatments for hyperlipidaemia, atherosclerosis and other vascular conditions that provide more efficient delivery of treatment.

### SUMMARY OF THE INVENTION

In one embodiment, the present invention provides a composition comprising: (a) at least one selective CB₁ receptor antagonist; and (b) at least one cholesterol lowering compound.

Therapeutic combinations also are provided comprising: (a) a first amount of at least one selective CB₁ receptor antagonist; and (b) a second amount of at least one cholesterol lowering compound, wherein the first amount and the second amount together comprise a therapeutically effective amount for the treatment or prevention of a vascular condition, diabetes, obesity, hyperlipidemia, metabolic syndrome, or lowering a concentration of a sterol in plasma of a subject.

Pharmaceutical compositions for the treatment or prevention of a vascular condition, diabetes, obesity, hyperlipidemia, metabolic syndrome, or lowering a concentration of a sterol in plasma of a subject, comprising a therapeutically effective amount of the above compositions or therapeutic combinations and a pharmaceutically acceptable carrier also are provided.

Methods of treatment or prevention of a vascular condition, diabetes, obesity, hyperlipidemia, metabolic syndrome, or lowering a concentration of a sterol in plasma of a subject, comprising the step of administering to a mammal in need of such treatment an effective amount of the above compositions or therapeutic combinations also are provided.

Other than in the operating examples, or where otherwise indicated, all numbers expressing quantities of ingredients, reaction conditions, and so forth used in the specification and claims are to be understood as being modified in all instances by the term “about.”

### DETAILED DESCRIPTION

The compositions and therapeutic combinations of the present invention comprise at least one selective CB₁ receptor antagonist, and at least one cholesterol lowering compound.

In another embodiment, the compositions and combinations of the present invention comprise at least one selective CB1 receptor antagonist, and at least one sterol absorption inhibitor or at least one 5α-stanol absorption inhibitor.

In yet another embodiment of the present invention, there is provided a therapeutic combination comprising: (a) a first amount of at least one selective CB1 receptor antagonist; and (b) a second amount of at least one cholesterol lowering compound; wherein the first amount and the second amount together comprise a therapeutically effective amount for the treatment or prevention of one or more of a vascular condition, diabetes, obesity, metabolic syndrome, or lowering a concentration of a sterol in plasma of a subject.

In yet another embodiment, the present invention provides for a pharmaceutical composition for the treatment or prevention of one or more of a vascular condition, diabetes, obesity, metabolic syndrome, or lowering a concentration of a sterol in plasma of a subject, comprising a therapeutically effective amount of a composition or therapeutic combination comprising: (a) at least one selective CB1 receptor antagonist; (b) a cholesterol lower compound; and (c) a pharmaceutically acceptable carrier.

In yet another embodiment, the present invention provides for a method of treating or preventing one or more of a vascular condition, diabetes, obesity, metabolic syndrome, or lowering a concentration of a sterol in plasma of a subject, comprising the step of administering to a mammal in need of such treatment an effective amount of a composition or therapeutic combination comprising: (a) at least one selective CB1 receptor antagonist; (b) a cholesterol lowering compound; and (c) a pharmaceutically acceptable carrier.

The selective CB₁ receptor antagonist compounds of the present invention are selective CB₁ receptor antagonists of mammalian CB₁ receptors, preferably human CB, receptors, and variants thereof. Mammalian CB₁ receptors also include CB₁ receptors found in rodents, primates, and other mammalian species.

The selective CB₁ receptor antagonist compounds of the present invention are selective CB₁ receptor antagonists that bind to a CB₁ receptor with a binding affinity (K_{i(CB1)}, measured as described herein) of about 100 nM or less, preferably about 50 nM or less, more preferably, about 10 nM or less, even more preferably about 1 nM or less. These ranges are inclusive of all values and subranges therebetween.

The selective CB₁ receptor antagonist compounds of the present invention are selective CB₁ receptor antagonists that have a ratio of CB₁ receptor affinity to CB₂ receptor affinity (K_{i(CB1)}:K_{i(CB2)}, measured as described herein) of about 1:2 or better, preferably about 1:25 or better, more preferably about 1:50 or better, even more preferably about 1:75 or better, still more preferably about 1:100 or better, a even still more preferably about 1:120 or better. These ranges are inclusive of all values and subranges therebetween.

Thus, as described above, a selective CB₁ receptor antagonist of the present invention has an affinity for the CB₁ receptor, measured as described herein, of at least 100 nM or less, and a ratio of CB₁ to CB₂ receptor affinity (i.e., K_{i(CB1)}:K_{i(CB2)}) of at least 1:2 or better. Preferably, the CB₁ affinity is about 50 nM or less, and the K_{i(CB1)}:K_{i(CB2)} is about 1:25 or better. More preferably, the CB₁ affinity is about 10 nM or less, and the K_{i(CB1)}:K_{i(CB2)} is about 1:50 or better. Even more preferably, the CB₁ affinity is about 10 nM or less, and the K_{i(CB1)}:K_{i(CB2)} is about 1:75 or better. Most preferably, the CB₁ affinity is about 1 nM or less, and the K_{i(CB1)}:K_{i(CB2)} is about 1:120 or better. These ranges are inclusive of all values and subranges therebetween.

The selective CB₁ receptor antagonist can be administered in a therapeutically effective amount and manner to treat the specified condition. The daily dose of the selective CB₁ receptor antagonist(s) administered to a mammalian patient or subject can range from about 1 mg/kg to about 50 mg/kg (where the units mg/kg refer to the amount of selective CB₁ receptor antagonist per kg body weight of the patient), preferably about 1 mg/kg to about 25 mg/kg, more preferably about 1 mg/kg to about 10 mg/kg.

Alternatively, the daily dose can range from about 1 mg to about 50 mg, preferably about 1 mg to about 25 mg, more preferably about 5 mg to about 20 mg. Although a single administration of the selective CB₁ receptor antagonist can be efficacious, multiple dosages can also be administered. The exact dose, however, can readily be determined by the attending clinician and will depend on such factors as the potency of the compound administered, the age, weight, condition and response of the patient.

Selective CB₁ receptor antagonists according to the present invention include pyrazole derivatives, for example those described in U.S. Pat. Nos. 5,624,941, 6,344,474, 6,432,984, 6,028,084, 6,509,367, U.S. published patent application 2004/0039024, WO 98/43635, WO 01/32663, WO 03/020217, Lan et al., J. Med. Chem., 1999, vol. 42, 769-776; dihydropyrazole derivatives, for example those described in U.S. Pat. No. 6,476,060, WO 02/076949, WO 03/026647, and WO 03/026648; terphenyl derivatives, for example those described in WO 03/084943; diphenylpyridine derivatives, for example those described in WO 03/084930; long chain polyunsaturated fatty acids, for example those described in WO 2004/012727; substituted amides, for example those described in WO 03/077847, WO 03/086288, WO 03/082190, and WO 03/087037; substituted azetidines, for example those described in U.S. Pat. Nos. 6,355,631, 6,479,479, and 6,566,356, and WO 00/15609; pyrazine derivatives, for example those described in WO 03/051850 and WO 03/051851; arylsulfonamide derivatives, for example those described in U.S. Pat. Nos. 6,469,054 and 6,727,279, and U.S. published patent application 2003/073727; substituted pyrroles, bicyclic or tricyclic compounds, or imidazoles, for example those described in U.S. Pat. No. 6,653,304, WO 03/063781, WO 03/007887, and WO 03/027076; substituted heterocyclic derivatives, for example those described in U.S. published patent application 2004/0063700; substituted triazoles, for example those described in WO 03/082833; aryl benzothiophenes and aryl benzofurans, for example those described in U.S. Pat. No. 5,596,106 and WO 9602248; benzodioxoles, for example those described in WO 2004/013120; substituted pyrimidines, for example those described in WO 2004/029204; substituted furopyridine derivatives, for example those described in WO 2004/012671; substituted diphenylpyridines, for example those described in WO 03/082191; and thiazole derivatives, for example those described in WO 03/078413. All of the above patents, published patent applications, and journal articles are incorporated herein by reference in their entirety, including the chemical structures and methods of preparing the CB₁ antagonist compounds described therein.

The pyrazole derivatives useful in the practice of the present invention include compounds of formula A, or pharmaceutically acceptable salts, solvates, or esters thereof:

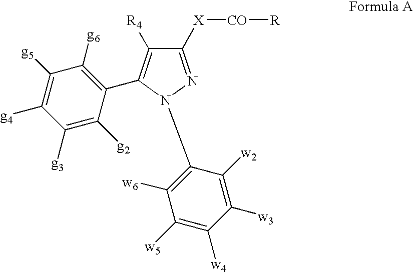

in which:

g₂, g₃, g₄, g₅ and g₆ and w₂, w₃, w₄, w₅ and w₆ are identical or different and are independently hydrogen, a chlorine or bromine atom, a (C₁-C₃)alkyl, a (C₁-C₃)alkoxy, a trifluoromethyl or a nitro group and g₄ is optionally a phenyl group;

R₄ is hydrogen or a (C₁-C₃)alkyl;

X is either a direct bond or a group —(CH₂)ₓN(R₃)—, in which R₃ is hydrogen or a (C₁-C₃)alkyl and x is zero or one; and

R is a group —NR₁R₂ in which R₁ and R₂ are independently a (C₁-C₆)alkyl; an optionally-substituted non-aromatic (C₃-C₁₅) carbocyclic radical; an amino(C₁-C₄) alkyl group in which the amino is optionally disubstituted by a (C₁-C₃) alkyl; a cycloalkyl(C₁-C₃)alkyl in which the cycloalkyl is C₃-C₁₂; a phenyl which is unsubstituted or monosubstituted or polysubstituted by a halogen, by a (C₁-C₅)alkyl or by a (C₁-C₅)alkoxy; a phenyl (C₁-C₃)alkyl; a diphenyl(C₁-C₃)alkyl; a naphthyl; an anthracenyl; a saturated 5- to 8-membered heterocyclic radical which is unsubstituted or substituted by a (C₁-C₃)alkyl, by a hydroxyl or by a benzyl group; a 1-adamantylmethyl; an aromatic heterocycle unsubstituted or mono-or-polysubstituted by a halogen, a (C₁-C₅)alkyl, a (C₁-C₅)alkoxy; a (C₁-C₃)alkyl substituted by an aromatic heterocycle unsubstituted or mono- or -polysubstituted by a halogen, a (C₁-C₅)alkyl, a (C₁-C₅)alkoxy, or else R₁ is hydrogen and R₂ is as defined above, or else R₁ and R₂, together with the nitrogen atom to which they are bonded, form a saturated 5- to 8-membered heterocyclic radical, said heterocyclic radical being other than morpholine when w₂, w₃, W₄, w₅ and w₆ and g₂, g₃, g₄, g₅ and g₆ are all hydrogen;

a group R₂ as defined above when X is —(CH₂)ₓ N(R₃)—; or

a group R₅ when X is a direct bond, R₅ being a (C₁-C₃)alkyl; a (C₃-C₁₂)cycloalkyl which is unsubstituted or substituted by a (C₁-C₅)alkyl; a phenyl(C₁-C₃)-alkyl which is unsubstituted or substituted by a halogen or by a (C₁-C₅)-alkyl; a cycloalkyl-(C₁-C₃)-alkyl in which the cycloalkyl is C₃-C₁₂ and is unsubstituted or substituted by a (C₁-C₅)alkyl; or a 2-norbornylmethyl; or one of their salts, where appropriate.

The non-aromatic C₃-C₁₅ carbocyclic radicals include saturated or unsaturated, fused or bridged monocyclic or polycyclic radicals, optionally terpene radicals. These radicals are optionally mono- or polysubstituted, said substituent(s) being different from a substituted carbonyl group. Advantageously, the monocyclic radicals are substituted by at least one group selected among the (C₁-C₅) alkyl, (C₁-C₅)alkoxy, halogen or hydroxy groups, it being understood that in the case of terpenes or terpene radicals, for example bornyl, menthyl or menthenyl, the alkyl groups of the terpene are not considered as substituents.

The monocyclic radicals include cycloalkyls, for example cyclopropyl, cyclopentyl, cyclohexyl, cycloheptyl, cyclooctyl and cyclododecyl, which are unsubstituted or substituted by at least one (C₁-C₅)-alkyl, (C₁-C₅)-alkoxy, halogen or hydroxy groups.

The fused, bridged or spiranic dicyclic or tricyclic radicals include for example norbornyl, bornyl, isobornyl, noradamantyl, adamantyl and spiro[5,5]undecanyl, said radicals being unsubstituted or substituted by a (C₁-C₅)-alkyl.

Saturated 5- to 8-membered heterocyclic radical is understood as meaning a fused or bridged, non-aromatic monocyclic, dicyclic or tricyclic heterocyclic radical, the heteroatom being S, O or N, or a non-aromatic monocyclic heterocyclic radical containing a nitrogen atom and an oxygen or sulfur atom, said radicals being for example tetrahydrofuranyl, tetrahydrothiofuranyl, tropyl, morpholinyl, thiomorpholinyl, piperidinyl, piperazinyl, pyrrolidinyl or quinuclidinyl, the 1-pyrrolidinyl, 1-piperidinyl, 1-hexahydroazepinyl, 4-morpholinyl and 4-thiomorpholinyl radicals being advantageous.

The aromatic heterocycles can be monocyclic or dicyclic, for example pyrrolyl, pyridyl, indolyl, quinolinyl, thiazolyl or isoindazolyl, these aromatic heterocycles being unsubstituted or substituted for example by halogens, (C₁-C₅)alkyl or (C₁-C₅)alkoxy. The preferred aromatic heterocycles are pyridyl, pyrrole, indole groups, the radicals 2-indolyl or 3-indolyl are particularly preferred.

In formula A above, preferably at least one of the substituents w₂, W₃, W₄, W₅ and w₆ and g₂, g₃, g₄, g₅ and g₆ is other than hydrogen.

In formula A above, when R is a group —NR₁R₂, preferably:

R₁ is hydrogen or a (C₁-C₆)alkyl group and R₂ is as defined above for (I); or

R₁ and R₂ are each a (C₁-C₆)alkyl group or a (C₃-C₆)cycloalkyl group; or

R₁ is hydrogen or a (C₁-C₆)alkyl group and R₂ is a cycloalkyl(C₁-C₃)alkyl group in which the cycloalkyl is C₃-C₁₂; a non-aromatic (C₃-C₁₅) carbocyclic radical which is unsubstituted or substituted as above mentioned; a phenyl which is unsubstituted or monosubstituted or polysubstituted by a halogen, by a (C₁-C₃)alkyl or by a (C₁-C₃)alkoxy; a phenyl-(C₁-C₃)alkyl or a (C₁-C₃)alkyl substituted by a 2- or 3-indolyl.

Particularly preferably, when R in formula A is a group —NR₁R₂, R₁ is hydrogen or a (C₁-C₆)alkyl and R₂ is a non-aromatic (C₃-C₁₅) carbocyclic radical, a cycloalkyl-(C₁-C₃)alkyl in which the cycloalkyl is C₃-C₆, or a 2- or 3-indolyl-(C₁-C₃)alkyl.

The preferred alkyl groups are methyl, ethyl, propyl and isopropyl.

In formula A above, R is advantageously a group —NR₁R₂ preferably selected from the radicals (1) to (74) below.

When R₁ and R₂, with the nitrogen atom to which they are bonded, are a heterocyclic, radical, this is preferably a 5-, 6- or 7-membered saturated heterocycle and can contain another heteroatom, especially oxygen or sulfur, for example a pyrrolidine, a piperidine, a hexahydroazepine, a morpholine or a thiomorpholine, with the limitation specified above.

The radicals represented by R as defined for formula A are preferably radicals selected from:
(1) propylamino
(2) butylamino
(3) isopropylamino
(4) dipentylamino
(5) 2-(N,N-diethylamino)ethylamino
(6) benzylamino
(7) 2-phenylethylamino
(8) 3-phenylpropylamino
(9) 3,3-diphenylpropylamino
(10) phenylamino
(11) 3-chlorophenylamino
(12) 4-methylphenylamino
(13) cyclopropylamino
(14) cyclopentylamino
(15) cyclohexylamino
(16) cycloheptylamino
(17) cyclooctylamino
(18) cyclododecylamino
(19) 2-methylcyclohexylamino
(20) 3-methylcyclohexylamino
(21) cis-4-methylcyclohexylamino
(22) trans-4-methylcyclohexylamino
(23) cis-4-tert-butylcyclohexylamino
(24) trans-4-tert-butylcyclohexylamino
(25) 4-hydroxycyclohexylamino
(26) 2-methoxycyclohexylamino
(27) 4-ethylcyclohexylamino
(28) 2,6-dimethylcyclohexylamino
(29) N-methylcyclohexylamino
(30) N,N-dicyclohexylamino
(31) endo-2-norbornylamino (or endo-bicyclo[2.2.1]-heptan-2-amino)
(32) exo-2-norbornylamino (or exo-bicyclo[2.2.1]heptan-2-amino)
(33) 1-adamantylamino
(34) 2-adamantylamino
(35) 1-noradamantylamino
(36) (1R)-bornylamino
(37) (1R)-isobornylamino
(38) spiro[5.5]undecanylamino
(39) cyclohexylmethylamino
(40) 1-adamantylmethylamino
(41) (2-tetrahydrofuranyl)methylamino
(42) 2-(N-methyl-2-pyrrolyl)ethylamino
(43) 2-(2-pyridinyl)ethylamino
(44) (2-indolyl)methylamino
(45) N-methyl(2-indolyl)methylamino
(46) 2-(3-indolyl)ethylamino
(47) N-methyl-2-(3-indolyl)ethylamino
(48) 4-(N-benzylpiperidinyl)amino
(49) 3-quinuclidylamino
(50) exo-bicyclo[3.2.1]octan-2-amino
(51) bicyclo[2.2.2]octan-2-amino
(52) 3-chlorobicyclo[3.2.1]oct-3-en-2-amino
(53) bicyclo[2.2.2]oct-2-en-5-amino
(54) exo-bicyclo[3.2.1]octan-3-amino
(55) endo-bicyclo[3.2.1]octan-3-amino
(56) endo-7-oxabicyclo[2.2.1]heptan-2-amino
(57) exo-7-oxabicyclo[2.2.1]heptan-2-amino
(58) endo-tricyclo[5.2.1.0.sup.2,6]decan-8-amino
(59) N-ethyl-1-adamantylamino
(60) tricyclo[2.2.1.0.sup.2,6]heptan-3-amino
(61) bicyclo[3.3.1]nonan-9-amino
(62) endo-1,3,3-trimethylbicyclo[2.2.1]heptan-2-amino (or fenchylamino)
(63) (1R, 2S-endo)-(+)-bicyclo[2.2.1]heptan-2-amino
(64) (1R,2R-exo)-(−)-bicyclo[2.2.1]heptan-2-amino
(65) (1S,2R-endo)-(−)-bicyclo[2.2.1]heptan-2-amino
(66) (1S,2S-exo)-(+)-bicyclo[2.2.1]heptan-2-amino
(67) 1-piperidinylamino
(68) 1-pyrrolidinylamino
(69) 1-hexahydroazepinylamino
(70) 4-morpholinylamino
(71) 4-thiomorpholinylamino
(72) N-methyl-exo-bicyclo[2.2.1]heptan-2-amino
(73) N-ethyl-exo-bicyclo[2.2.1]heptan-2-amino
(74) N-propyl-exo-bicyclo[2.2.1]heptan-2-amino.

A particularly preferred compound according to formula A is the pyrazole compound of formula A-1 (i.e., rimonabant), or pharmaceutically acceptable salts or solvates thereof:

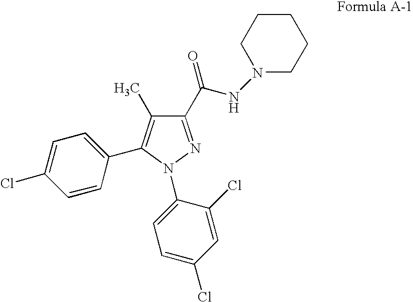

The pyrazole derivatives useful in the practice of the present invention also include compounds of formula B, or pharmaceutically acceptable salts, solvates, or esters thereof:

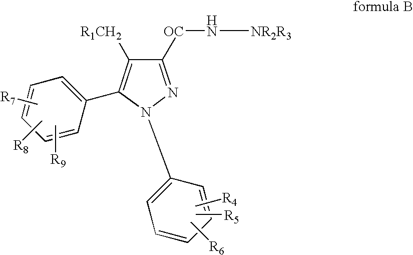

in which:

R₁ is hydrogen, a fluorine, a hydroxyl, a (C₁-C₅)alkoxy, a (C₁-C₅)alkylthio, a hydroxy(C₁-C₅)alkoxy, a group —NR₁₀R₁₁, a cyano, a (C₁-C₅)alkylsulfonyl or a (C₁-C₅)alkylsulfinyl;

R₂ and R₃ are a (C₁-C₄)alkyl or, together with the nitrogen atom to which they are bonded, form a saturated or unsaturated 5- to 10-membered heterocyclic radical which is unsubstituted or monosubstituted or polysubstituted by a (C₁-C₃)alkyl or by a (C₁-C₃)alkoxy;

R₄, R₅, R₆, R₇, R₈ and R₉ are each independently hydrogen, a halogen or a trifluoromethyl, and if R₁ is a fluorine, R₄, R₅, R₆, R₇, R₈ and/or R₉ can also be a fluoromethyl, with the proviso that at least one of the substituents R₄ or R₇ is other than hydrogen; and

R₁₀ and R₁₁, are each independently hydrogen or a (C₁-C₅)alkyl, or R₁₀ and R₁₁, together with the nitrogen atom to which they are bonded, form a heterocyclic radical selected from pyrrolidin-1-yl, piperidin-1-yl, morpholin-4-yl and piperazin-1-yl, which is unsubstituted or substituted by a (C₁-C₄)alkyl,

and their pharmaceutically acceptable salts, solvates, or esters.

The dihydropyrazole derivatives useful in the practice of the present invention include compounds of formula C, or pharmaceutically acceptable salts, solvates, or esters thereof: 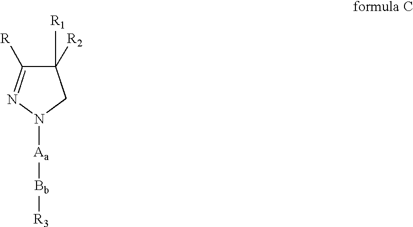
wherein:

R represents phenyl, thienyl or pyridyl, each of which is unsubstituted or substituted with 1, 2 or 3 substituents Y, which are the same or different and are chosen from (C₁₋₃)alkyl, (C₁₋₃)alkoxy, hydroxy, halogen, trifluoromethyl, trifluoromethylthio, trifluoromethoxy, nitro, amino, monoalkyl (C₁₋₂)amino, dialkyl(C₁₋₂)amino, monoalkyl(C₁₋₂)amido, dialkyl(C₁₋₂)amido, (C₁₋₃)-alkyl sulfonyl, dimethylsulfamido, (C₁₋₃)alkoxycarbonyl, carboxyl, trifluoromethylsulfonyl, cyano, carbamoyl, sulfamoyl and acetyl; or

R represents naphthyl;

R₁ represents phenyl, thienyl or pyridyl, each of which is unsubstituted or substituted with 1, 2 or 3 substituents Y, which are the same or different and are chosen from (C₁₋₃)alkyl, (C₁₋₃)alkoxy, hydroxy, halogen, trifluoromethyl, trifluoromethylthio, trifluoromethoxy, nitro, amino, monoalkyl(C₁₋₂)amino, dialkyl(C₁₋₂)amino, monoalkyl (C₁₋₂)amido, dialkyl(C₁₋₂)amido, (C₁₋₃)alkyl sulfonyl, dimethylsulfamido, (C₁₋₃)alkoxycarbonyl, carboxyl, trifluoromethylsulfonyl, cyano, carbamoyl, sulfamoyl and acetyl; or

R₁ represents naphthyl;

R₂ represents hydrogen, hydroxy, (C₁₋₃)alkoxy, acetyloxy or propionyloxy;

Aₐ represents one of the groups (i), (ii), (iii), (iv) or (v): 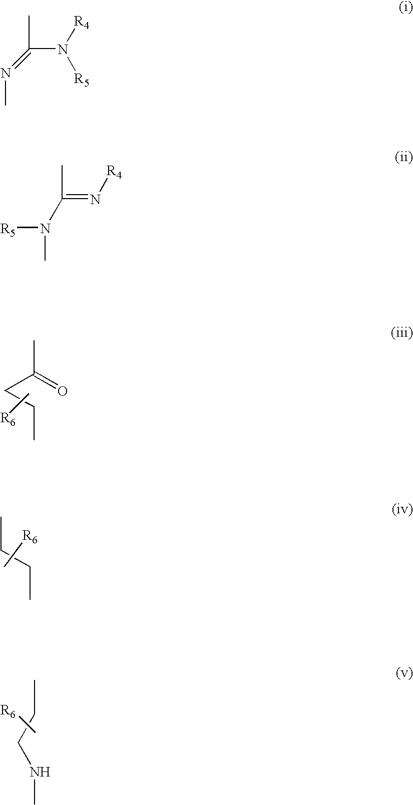
wherein

R₄ represents hydrogen, (C₁₋₈) branched or unbranched alkyl or (C₃₋₈) cycloalkyl; and when R₅ represents hydrogen, R₄ optionally further represents acetamido, dimethylamino, 2,2,2-trifluoroethyl, phenyl or pyridyl;

R₅ represents hydrogen, (C₁₋₈) branched or unbranched alkyl or (C₃₋₈) cycloalkyl;

R₆ represents hydrogen or (C₁₋₃) unbranched alkyl;

B_{b} represents sulfonyl or carbonyl; and

R₃ represents benzyl, phenyl, thienyl or pyridyl, each of which is unsubstituted or substituted with 1, 2 or 3 substituents Y, which are the same or different, or R₃ represents (C₁₋₈) branched or unbranched alkyl or (C₃₋₈) cycloalkyl, or R₃ represents naphthyl.

The dihydropyrazole derivatives useful in the practice of the present invention also include compounds of formula D, or pharmaceutically acceptable salts, solvates, or esters thereof:

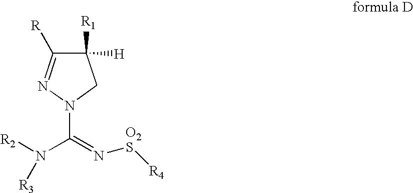

wherein R and R₁ are the same or different and represent 3-pyridyl or 4-pyridyl, or phenyl which may be substituted with halogen or methoxy;

R₂ and R₃ are the same or different and represent hydrogen, alkyl (C₁₋₃) or dimethylamino; and

R₄ represents phenyl which may be substituted with 1, 2 or 3 substituents selected from the group halogen, trifluoromethyl, methoxy and (C₁₋₃)alkyl.

The dihydropyrazole derivatives useful in the practice of the present invention also include compounds of formula E, or pharmaceutically acceptable salts, solvates, or esters thereof:

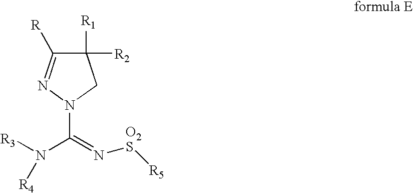

wherein:

R and R₁ independently represent phenyl, thienyl or pyridyl which groups may be substituted with 1, 2, 3 or 4 substituents Y, which can be the same or different, from the group (C₁₋₃)-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, (C₁₋₃)alkoxycarbonyl, carboxyl, trifluoromethylsulfonyl, cyano, carbamoyl, sulfamoyl and acetyl, or R and/or R₁ represent naphthyl;

R₂ represents hydrogen, hydroxy, (C₁₋₃)-alkoxy, acetyloxy or propionyloxy,

R₃ represents a hydrogen atom or a branched or unbranched (C₁₋₈) alkyl group or a (C₃₋₇)cycloalkyl group which alkyl group or cycloalkyl group may be substituted with a hydroxy group;

R₄ represents a C₂₋₁₀ branched or unbranched heteroalkyl group, C₃₋₈ non aromatic heterocycloalkyl group or C₄₋₁₀ non-aromatic heterocycloalkyl-alkyl group which groups contain one or more heteroatoms from the group (O. N. S) or a —SO₂— group, which C₂₋₁₀ branched or unbranched heteroalkyl group, C₃₋₈ non aromatic heterocycloalkyl group or C₄₋₁₀ non-aromatic heterocycloalkyl-alkyl group may be substituted with a keto group, trifluoromethyl group, (C₁₋₃)alkyl group, hydroxy, amino, monoalkylamino, or dialkylamino group or a fluoro atom; or R₄ represents an amino, hydroxy, phenoxy or benzyloxy group; or R₄ represents a (C₁₋₈)alkoxy, (C₃₋₈)alkenyl, (C₅₋₈)cycloalkenyl or (C₁₋₉)cycloalkenylalkyl group which groups may contain a sulphur, nitrogen or oxygen atom, a keto group or —SO₂— group, which alkoxy, alkenyl and cycloalkenyl groups may be substituted with a hydroxy group, a trifluoromethyl group, an amino group, a monoalkylamino group or dialkylamino group or a fluoro atom; or R₄ represents a (C₂₋₅)alkyl group which alkyl group contains a fluoro atom; or R₄ represents an imidazolylalkyl group, benzyl, pyridylmethyl, phenethyl or thienyl group, or R₄ represents a substituted phenyl, benzyl, pyridyl, thienyl, pyridylmethyl or phenethyl group wherein the aromatic rings are substituted with 1, 2 or 3 of the substituents Y. wherein Y has the meaning as indicated above; or when R₃ is H or methyl, R₄ may represent a group NR₆R₇ wherein

R₆ and R₇ are the same or different and represent (C₂₋₄)alkyl, (C₂₋₄)trifluoroalkyl or R₆ represents a methyl group with the proviso that R₇ represents a (C₂₋₄)alkyl group, or R₆ and R₇— together with the nitrogen atom to which they are bonded—form a saturated or unsaturated heterocyclic moiety having 4 to 8 ring 15 atoms which heterocyclic moiety may contain an oxygen or sulphur atom or a keto group or —SO₂— group or an additional nitrogen atom, which saturated or unsaturated heterocyclic moiety may be substituted with a (C₁₋₄)alkyl group, or

R₃ and R₄ together with the nitrogen atom to which they are bonded form a saturated or unsaturated, monocyclic or bicyclic heterocyclic moiety having 4 to 10 ring atoms, which heterocyclic moiety may contain one or more atoms from the group (O, N, S) or a keto group or —SO₂— group, which moiety may be substituted with a (C₁₋₄)alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinylorhexahydro-1H-azepinyl group,

R₅ represents benzyl, phenyl, thienyl or pyridyl which may be substituted with 1, 2, 3 or 4 substituents Y, wherein Y has the meaning as indicated above, which can be the same or different, or R₅ represents C₁₋₈ branched or unbranched alkyl, C₃₋₈ alkenyl, C₃₋₁₀ cycloalkyl, C₅₋₁₀ bicycloalkyl, C₆₋₁₀ tricycloalkyl or C₅₋₈ cycloalkenyl or R₅ represents naphthyl.

The dihydropyrazole derivatives useful in the practice of the present invention also include compounds of formulae F1 or F2, or pharmaceutically acceptable salts, solvates, or esters thereof: 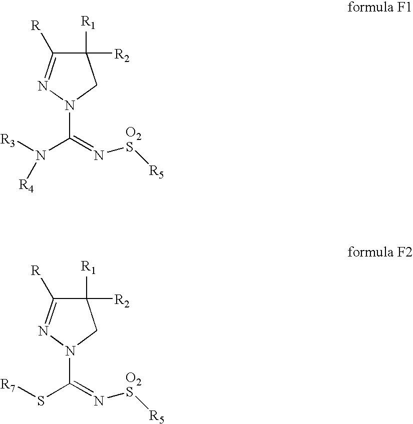
wherein:

R and R₁ independently represent phenyl, thienyl or pyridyl which groups may be substituted with 1, 2 or 3 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R represent naphthyl,

R₂ represents hydrogen, hydroxy, C₁₋₃-alkoxy, acetyloxy or propionyloxy;

R₃ represents a hydrogen atom or a branched or unbranched C₁₋₈ alkyl group or a C₃₋₇ cycloalkyl group which alkyl group or cycloalkyl group may be substituted with a hydroxy group;

R₄ represents a hydrogen atom or a branched or unbranched C₁₋₈ alkyl, C₃₋₈ cycloalkyl, C₂₋₁₀ heteroalkyl, C₃₋₈ nonaromatic heterocycloalkyl or C₄-10 nonaromatic heterocycloalkyl-alkyl moiety which moieties may contain one or more heteroatoms from the group (O, N, S), which moieties may be substituted with a keto group, trifluoromethyl group, C₁₋₃ alkyl group, hydroxy, amino, monoalkylamino, or dialkylamino group or a fluoro atom, or R₄ represents an amino, hydroxy, phenoxy or benzyloxy group or R₄ represents a branched or unbranched C₁₋₈ alkoxy, C₃₋₈ alkenyl, C5-8 cycloalkenyl or C6-9 cycloalkenylalkyl group which groups may contain a sulphur, nitrogen or oxygen atom, a keto group or —SO₂— group which C1-8 alkoxy, C₃₋₈ alkenyl, C5-8 cycloalkenyl or C6-9 cycloalkenylalkyl groups may be substituted with a hydroxy group, a trifluoromethyl group, an amino group, a monoalkylamino group or dialkylamino group or a fluoro atom, or R₄ represents a phenyl, benzyl, pyridyl, thienyl, pyridylmethyl or phenethyl group wherein the aromatic rings may be substituted with 1, 2 or 3 of the substituents Y, wherein Y has the meaning as indicated above, or

R₄ represents a group NR₈R₉ with the proviso that R₃ represents a hydrogen atom or a methyl group and wherein R₈ and R₉ are the same or different and represent C₁₋₄ alkyl or C₂₋₄ trifluoroalkyl or R₈ and R₉—together with the nitrogen atom to which they are bonded—form a saturated or un-saturated heterocyclic moiety having 4 to 8 ring atoms which heterocyclic moiety may contain an oxygen or sulphur atom or a keto group or —SO₂— group or an additional nitrogen atom, which saturated or unsaturated heterocyclic moiety may be substituted with a C₁₋₄ alkyl group or

R₃ and R₄—together with the nitrogen atom to which they are bonded—form a saturated or unsaturated, monocyclic or bicyclic heterocyclic moiety having 4 to 10 ring atoms, which heterocyclic moiety may contain one or more atoms from the group (O, N, S) or a keto group or —SO₂— group, which moiety may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group;

R₅ and R₆ independently of each other represent a hydrogen atom or a branched or unbranched C₁₋₈ alkyl or alkenyl group which groups may contain one or more heteroatoms from the group (O, N, S), a keto group or a —SO₂-group and which groups may be substituted with a hydroxy or amino group, or R₅ and R₆ independently of each other represent a C₃₋₈ cycloalkyl group or C₃₋₈ cycloalkenyl group which may contain one or more ring heteroatoms from the group (O, N, S) or the —SO₂— group and which groups may be substituted with a hydroxy group, alkyl (C₁₋₃), the —SO₂— group, the keto group, amino group, monoalkylamino group (C₁₋₃) or dialkylamino group (C₁₋₃), or R₅ represents a naphthyl group or a phenyl group which phenyl group may be substituted with 1, 2 or 3 substituents Y wherein Y has the meaning as described hereinabove, with the proviso that R₆ represents a hydrogen atom, or a branched or unbranched alkyl group (C1-5) which alkyl group may contain one or more heteroatoms from the group (O, N, S) or the —SO₂— group and which alkyl group may be substituted with a hydroxy, keto or amino group, or

R₅ and R₆— together with the nitrogen atom to which they are bonded—form a monocyclic, bicyclic or tricyclic alkyl or alkenyl group which may contain ring heteroatoms from the group (O, N, S), the keto or the SO₂ group and which monocyclic, bicyclic or tricyclic alkyl or alkenyl group may be substituted with a hydroxy group, alkyl (C₁₋₃) group, SO₂ group, keto group, amino group, monoalkylamino group (C₁₋₃), dialkylamino group (C₁₋₃), pyrrolidinyl group or piperidinyl group, which monocyclic, bicyclic or tricyclic alkyl or alkenyl group may contain an annelated phenyl group which annelated phenyl group may be substituted with 1 or 2 substituents Y. wherein Y has the meaning as described herein above, and

R₇ represents branched or unbranched C₁₋₃ alkyl.

The term “therapeutically effective amount” means that amount of therapeutic agents of the invention, such as the selective CB₁ receptor antagonist, substituted azetidinone(s) or substituted β-lactam(s) and other pharmacological or therapeutic agents described below, that will elicit a biological or medical response of a subject, tissue, system, animal or mammal that is being sought by the administrator (such as a researcher, doctor or veterinarian) which includes alleviation of the symptoms of the condition or disease being treated and the prevention, slowing or halting of progression of one or more conditions, for example vascular conditions, such as hyperlipidaemia (for example atherosclerosis, hypercholesterolemia or sitosterolemia), metabolic syndrome, vascular inflammation, stroke, diabetes, obesity and/or to reduce the level of sterol(s) (such as cholesterol) in the plasma.

As used herein, “combination therapy” or “therapeutic combination” means the administration of two or more therapeutic agents, such as a selective CB₁ receptor antagonist, substituted azetidinone(s) or substituted β-lactam(s), to prevent or treat a condition, for example a vascular condition, such as hyperlipidaemia (for example atherosclerosis, hypercholesterolemia or sitosterolemia), vascular inflammation, metabolic syndrome, stroke, diabetes, obesity and/or reduce the level of sterol(s) (such as cholesterol) in the plasma or tissue. As used herein, “vascular” comprises cardiovascular, cerebrovascular and combinations thereof. The compositions, combinations and treatments of the present invention can be administered by any suitable means which produce contact of these compounds with the site of action in the body, for example in the plasma, liver or small intestine of a subject (mammal or human or other animal). Such administration includes coadministration of these therapeutic agents in a substantially simultaneous manner, such as in a single tablet or capsule having a fixed ratio of active ingredients or in multiple, separate capsules for each therapeutic agent. Also, such administration includes the administration of each type of therapeutic agent in a sequential manner. In either case, the treatment using the combination therapy will provide beneficial effects in treating the condition. A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve patient compliance. Also, therapeutic agents can be selected to provide a broader range of complimentary effects or complimentary modes of action.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for illustration purposes, a compound of Formula I and an additional therapeutic agent, such as a selective CB₁ receptor antagonist, e.g., rimonabant, may be present in fixed amounts (dosage amounts) in a single dosage unit (e.g., a capsule, a tablet and the like). A commercial example of a single dosage unit containing fixed amounts of two different active compounds is VYTORIN® (available from Merck Schering-Plough Pharmaceuticals, Kenilworth, N.J.).

Alternatively, the combination therapy of the present invention may be administered in different dosage units. That is, the combination may be administered by sequential or concurrent administration of different dosage units, for example by administering a first dosage unit comprising ezetimibe, followed by a second dosage unit comprising rimonabant, by administering a first dosage unit comprising rimonabant, followed by a second dosage unit comprising ezetimibe, or by simultaneously administering a first dosage unit comprising ezetimibe, and a second dosage unit comprising rimonabant.

If formulated as a fixed dose, such combination products employ the therapeutic compositions or combinations of this invention within the dosage range described herein. For example, a selective CB₁ receptor antagonist and a compound of Formula I may also be administered sequentially with known therapeutic agents when a combination formulation is inappropriate. The invention is not limited in the sequence of administration; for example, compounds of Formula I may be administered either prior to or after administration of the selective CB₁ receptor antagonist. Such techniques are within the skills of persons skilled in the art as well as attending physicians.

As discussed above, the compositions, pharmaceutical compositions and therapeutic combinations of the present invention comprise: (a) one or more selective CB₁ receptor antagonists; and (b) one or more cholesterol lowering compounds. A non-limiting list of cholesterol lowering compounds useful in the present invention include HMG CoA reductase inhibitor compounds such as lovastatin (for example MEVACOR® which is available from Merck & Co.), simvastatin (for example ZOCOR® which is available from Merck & Co.), pravastatin (for example PRAVACHOL® which is available from Bristol Meyers Squibb), atorvastatin, fluvastatin, cerivastatin, CI-981, rivastatin (sodium 7-(4-fluorophenyl)-2,6-diisopropyl-5-methoxymethylpyridin-3-yl)-3,5-dihydroxy-6-heptanoate), rosuvastatin calcium (CRESTOR® from AstraZeneca Pharmaceuticals), pitavastatin (such as NK-104 of Negma Kowa of Japan); HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride); sterol biosynthesis inhibitors such as DMP-565; nicotinic acid derivatives (e.g., compounds comprising a pyridine-3-carboxylate structure or a pyrazine-2-carboxylate structure, including acid forms, salts, esters, zwitterions and tautomers) such as niceritrol, nicofuranose and acipimox (5-methyl pyrazine-2-carboxylic acid 4-oxide); clofibrate; gemfibrazol; bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof; inorganic cholesterol sequestrants such as bismuth salicylate plus montmorillonite clay, aluminum hydroxide and calcium carbonate antacids; ileal bile acid transport (“IBAT”) inhibitors (or apical sodium co-dependent bile acid transport (“ASBT”) inhibitors) such as benzothiepines, for example the therapeutic compounds comprising a 2,3,4,5-tetrahydro-1-benzothiepine 1,1-dioxide structure such as are disclosed in PCT Patent Application WO 00/38727 which is incorporated herein by reference; AcylCoA:Cholesterol O-acyltransferase (“ACAT”) Inhibitors such as avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea), and the compounds described in P. Chang et al., “Current, New and Future Treatments in Dyslipidaemia and Atherosclerosis”, Drugs 2000 July; 60(1); 55-93, which is incorporated by reference herein; Cholesteryl Ester Transfer Protein (“CETP”) Inhibitors such as those disclosed in PCT Patent Application No. WO 00/38721 and U.S. Pat. No. 6,147,090, which are incorporated herein by reference; probucol or derivatives thereof, such as AGI-1067 and other derivatives disclosed in U.S. Pat. Nos. 6,121,319 and 6,147,250, herein incorporated by reference; low-density lipoprotein (LDL) receptor activators such as HOE-402, an imidazolidinyl-pyrimidine derivative that directly stimulates LDL receptor activity, described in M. Huettinger et al., “Hypolipidemic activity of HOE-402 is Mediated by Stimulation of the LDL Receptor Pathway”, Arterioscler. Thromb. 1993; 13:1005-12, herein incorporated by reference; fish oils containing Omega 3 fatty acids (3-PUFA); natural water soluble fibers, such as psyllium, guar, oat and pectin; plant stanols and/or fatty acid esters of plant stanols, such as sitostanol ester used in BENECOL® margarine; and the substituted azetidinone or substituted β-lactam sterol absorption inhibitors discussed in detail below.

As used herein, “sterol absorption inhibitor” means a compound capable of inhibiting the absorption of one or more sterols, including but not limited to cholesterol, phytosterols (such as sitosterol, campesterol, stigmasterol and avenosterol), 5α-stanols (such as cholestanol, 5α-campestanol, 5α-sitostanol), and/or mixtures thereof, when administered in a therapeutically effective (sterol and/or 5α-stanol absorption inhibiting) amount to a mammal or human.

In one embodiment, substituted azetidinones useful in the compositions, therapeutic combinations and methods of the present invention are represented by Formula (I) below: 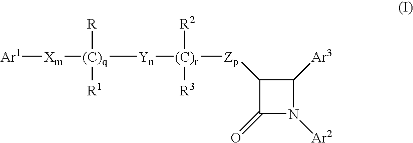
or pharmaceutically acceptable salts, solvates, or esters of the compounds of Formula (I), wherein, in Formula (I) above:

Ar¹ and Ar² are independently selected from the group consisting of aryl and R⁴-substituted aryl;

Ar³ is aryl or R⁵-substituted aryl;

X, Y and Z are independently selected from the group consisting of —CH2-, —CH(lower alkyl)- and —C(lower alkyl)₂—;

R and R² are independently selected from the group consisting of —OR⁶, —OC(O)R⁶, —OC(O)OR⁹ and —OC(O)NR⁶R⁷;

R¹ and R³ are independently selected from the group consisting of hydrogen, lower alkyl and aryl;

q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO₂R⁹, —COOR⁶, —CONR⁶R⁷, —COR⁶, —SO₂NR⁶R⁷, S(O)O₀₋₂R⁹, —O(CH₂)₁₋₁₀—COOR⁶, —O(CH₂)₁₋₁₀CONR⁶R⁷, -(lower alkylene)COOR⁶, —CH═CH—C(O)OR⁶, —CF₃, —CN, —NO₂ and halogen;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —CONR⁶R⁷, —C(O)R⁶, —SO₂N R⁶R⁷, S(O)₀₋₂R⁹, —O(CH₂)₁₋₁₀—C(O)OR⁶, —O(CH₂)₁₋₁₀C(O)NR⁶R⁷, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

Preferably, R⁴ is 1-3 independently selected substituents, and R⁵ is preferably 1-3 independently selected substituents.

As used herein, the term “alkyl” or “lower alkyl” means straight or branched alkyl chains having from 1 to 6 carbon atoms and “alkoxy” means alkoxy groups having 1 to 6 carbon atoms. Non-limiting examples of lower alkyl groups include, for example methyl, ethyl, propyl, and butyl groups.

“Alkenyl” means straight or branched carbon chains having one or more double bonds in the chain, conjugated or unconjugated. Similarly, “alkynyl” means straight or branched carbon chains having one or more triple bonds in the chain. Where an alkyl, alkenyl or alkynyl chain joins two other variables and is therefore bivalent, the terms alkylene, alkenylene and alkynylene are used.

“Cycloalkyl” means a saturated carbon ring of 3 to 6 carbon atoms, while “cycloalkylene” refers to a corresponding bivalent ring, wherein the points of attachment to other groups include all positional isomers.

“Halogeno” or “halogen” refers to fluorine, chlorine, bromine or iodine radicals.

“Aryl” means phenyl, naphthyl, indenyl, tetrahydronaphthyl or indanyl.

“Phenylene” means a bivalent phenyl group, including ortho-, meta- and para-substitution.

The statements wherein, for example, R, R¹, R² and R³, are said to be independently selected from a group of substituents, mean that R, R¹, R² and R³ are independently selected, but also that where an R, R¹, R² and R³ variable occurs more than once in a molecule, each occurrence is independently selected (e.g., if R is —OR⁶, wherein R⁶ is hydrogen, R² can be —OR⁶ wherein R⁶ is lower alkyl). Those skilled in the art will recognize that the size and nature of the substituent(s) will affect the number of substituents that can be present.

Certain compounds useful in the therapeutic compositions or combinations of the invention may have at least one asymmetrical carbon atom and therefore all isomers, including enantiomers, diastereomers, stereoisomers, rotamers, tautomers and racemates of the compounds of Formula (I-XI) (where they exist) are contemplated as being part of this invention. The invention includes d and l isomers in both pure form and in admixture, including racemic mixtures. Isomers can be prepared using conventional techniques, either by reacting optically pure or optically enriched starting materials or by separating isomers of a compound of the Formulae I-XI. Isomers may also include geometric isomers, e.g., when a double bond is present.

Those skilled in the art will appreciate that for some of the compounds of the Formulae I-XI, one isomer may show greater pharmacological activity than other isomers.

Compounds useful in the therapeutic compositions or combinations of the invention with an amino group can form pharmaceutically acceptable salts with organic and inorganic acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. The salt is prepared by contacting the free base form with a sufficient amount of the desired acid to produce a salt. The free base form may be regenerated by treating the salt with a suitable dilute aqueous base solution such as dilute aqueous sodium bicarbonate. The free base form differs from its respective salt form somewhat in certain physical properties, such as solubility in polar solvents, but the salt is otherwise equivalent to its respective free base forms for purposes of the invention.

Certain compounds useful in the therapeutic compositions or combinations of the invention are acidic (e.g., those compounds which possess a carboxyl group). These compounds form pharmaceutically acceptable salts with inorganic and organic bases. Examples of such salts are the sodium, potassium, calcium, aluminum, gold and silver salts. Also included are salts formed with pharmaceutically acceptable amines such as ammonia, alkyl amines, hydroxyalkylamines, N-methylglucamine and the like.

As used herein, “solvate” means a molecular or ionic complex of molecules or ions of solvent with those of solute (for example, one or more compounds of Formulae I-XI, isomers of the compounds of Formulae I-XI, or prodrugs of the compounds of Formulae I-XI). Non-limiting examples of useful solvents include polar, protic solvents such as water and/or alcohols (for example methanol).

Pharmaceutically acceptable esters of compounds useful in the therapeutic compositions or combinations of the invention include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

As used herein, “prodrug” means compounds that are drug precursors which, following administration to a patient, release the drug in vivo via some chemical or physiological process (e.g., a prodrug on being brought to the physiological pH or through enzyme action is converted to the desired drug form).

Preferred compounds of Formula (I) are those in which Ar¹ is phenyl or R⁴-substituted phenyl, more preferably (4-R⁴)-substituted phenyl. Ar² is preferably phenyl or R⁴-substituted phenyl, more preferably (4-R⁴)-substituted phenyl. Ar³ is preferably R⁵-substituted phenyl, more preferably (4-R⁵)-substituted phenyl. When Ar¹ is (4-R⁴)-substituted phenyl, R⁴ is preferably a halogen. When Ar² and Ar³ are R⁴- and R⁵-substituted phenyl, respectively, R⁴ is preferably halogen or —OR⁶ and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ wherein R⁶ is hydrogen, or a group readily metabolizable to a hydroxyl (such as —OC(O)R⁶, —OC(O)OR⁹ and —OC(O)NR⁶R⁷, defined above).

The sum of m, n, p, q and r is preferably 2, 3 or 4, more preferably 3.

Preferred are compounds wherein m, n and r are each zero, q is 1 and p is 2.

Also preferred are compounds of Formula (I) in which p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of Formula (I) wherein p, q and n are each zero, r is 1, m is 2, X is —CH₂— and R² is —OR⁶, especially when R⁶ is hydrogen.

Another group of preferred compounds of Formula (I) is that in which Ar¹ is phenyl or R⁴-substituted phenyl, Ar² is phenyl or R⁴-substituted phenyl and Ar³ is R⁵-substituted phenyl. Also preferred are compounds in which Ar¹ is phenyl or R⁴-substituted phenyl, Ar² is phenyl or R⁴-substituted phenyl, Ar³ is R⁵-substituted phenyl, and the sum of m, n, p, q and r is 2, 3 or 4, more preferably 3. More preferred are compounds wherein Ar¹ is phenyl or R⁴-substituted phenyl, Ar² is phenyl or R⁴-substituted phenyl, Ar³ is R⁵-substituted phenyl, and wherein m, n and r are each zero, q is 1 and p is 2, or wherein p, q and n are each zero, r is 1 and m is 2 or 3.

In a preferred embodiment, a substituted azetidinone of Formula (I) useful in the compositions, therapeutic combinations and methods of the present invention is represented by Formula (II) (ezetimibe) below: 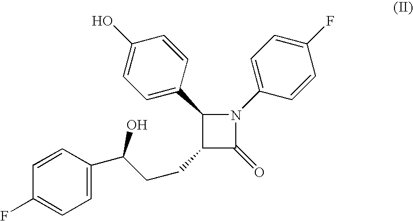
or pharmaceutically acceptable salts or solvates of the compound of Formula (II). The compound of Formula (II) can be in anhydrous or hydrated form. A product containing ezetimibe compound is commercially available as ZETIA® ezetimibe formulation from MSP Pharmaceuticals.

Compounds of Formula I can be prepared by a variety of methods well known to those skilled in the art, for example such as are disclosed in U.S. Pat. Nos. 5,631,365, 5,767,115, 5,846,966, 6,207,822, 6,627,757, 6,093,812, 5,306,817, 5,561,227, 5,688,785, and 5,688,787, each of which is incorporated herein by reference, and in the Example below.

Alternative substituted azetidinones useful in the compositions, therapeutic combinations and methods of the present invention are represented by Formula (III) below: 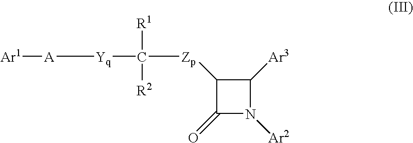
or a pharmaceutically acceptable salt thereof or a solvate thereof, or an ester thereof, wherein, in Formula (III) above:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(lower alkyl)₂—;

A is selected from —O—, —S—, —S(O)— or —S(O)₂—;

R¹ is selected from the group consisting of —OR⁶, —OC(O)R⁶, —OC(O)OR⁹ and —OC(O)NR⁶R⁷;

R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —OC(O)R⁶, —OC(O)OR⁹, —O(CH₂)₁₋₅OR⁹, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂-lower alkyl, —NR⁶S(O)₂-aryl, —C(O)NR⁶R⁷, —COR⁶, —SO₂NR⁶R⁷, S(O)₀₋₂-alkyl, S(O)₀₋₂-aryl, —O(CH₂)₁₋₁₀—C(O)OR⁶, —O(CH₂)₁₋₁₀C(O)NR⁶R⁷, o-halogeno, m-halogeno, o-lower alkyl, m-lower alkyl, -(lower alkylene)-C(O)OR⁶, and —CH═CH—C(O)OR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

Methods for making compounds of Formula III are well known to those skilled in the art. Non-limiting examples of suitable methods are disclosed in U.S. Pat. No. 5,688,990, which is incorporated herein by reference.

In another embodiment, substituted azetidinones useful in the compositions, therapeutic combinations and methods of the present invention are represented by Formula (IV): 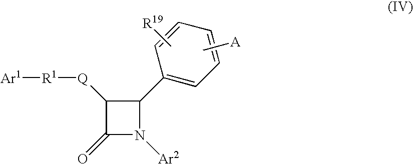
or a pharmaceutically acceptable salt thereof or a solvate thereof, or an ester thereof, wherein, in Formula (IV) above:

A is selected from the group consisting of R²-substituted heterocycloalkyl, R²-substituted heteroaryl, R²-substituted benzofused heterocycloalkyl, and R²-substituted benzofused heteroaryl;

Ar¹ is aryl or R³-substituted aryl;

Ar² is aryl or R⁴-substituted aryl;

Q is a bond or, with the 3-position ring carbon of the azetidinone, forms the spiro group 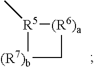
and

R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;

R⁵ is selected from:

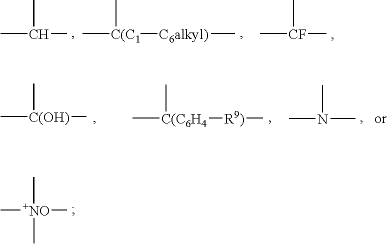

R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)═CH—, a is 1; provided that when R⁷ is —CH═CH— or —C(C₁-C₆ alkyl)═CH—, b is 1; provided that when a is 2 or 3, the R⁶'s can be the same or different; and provided that when b is 2 or 3, the R⁷'s can be the same or different;

and when Q is a bond, R¹ also can be selected from:

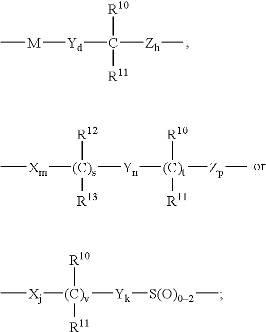

where M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);
R¹⁰ and R¹² are independently selected from the group consisting of —OR¹⁴, —OC(O)R¹⁴, —OC(O)OR¹⁶ and —OC(O)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is O and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —C(O)R¹⁴, hydroxy(C₁-C₆)alkyl, (C₁-C₆)alkoxy(C₁-C₆)alkyl, NO₂, —S(O)₀₋₂R¹⁶, —S(O)₂NR¹⁴R¹⁵ and —(C—C₆ alkylene)COOR¹⁴; when R² is a substituent on a heterocycloalkyl ring, R² is as defined, or R² is =0 or 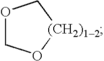
and, where R² is a substituent on a substitutable ring nitrogen, R² is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

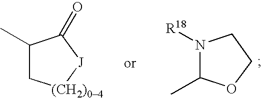

wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —OC(O)R⁴, —OC(O)OR⁶, —O(CH₂)₁₋₅OR¹⁴, —OC(O)NR⁴R⁵, —NR¹⁴R¹⁵, —NR¹⁴C(O)R¹⁵, —NR¹⁴C(O)OR¹⁶, —NR¹⁴C(O)NR¹⁵R¹⁹, —NR¹⁴S(O)₂R¹⁶, —C(O)OR⁴, —C(O)NR¹⁴R¹⁵, —C(O)R¹⁴, —S(O)₂NR⁴R⁵, S(O)₀₋₂R¹⁶, —O(CH₂)₁₋₁₀—COOR⁴, —O(CH₂)₁₋₁₀C(O)NR⁴R⁵, —(C₁-C₆ alkylene)-C(O)OR¹⁴, —CH═CH—C(O)OR¹⁴, —CF₃, —CN, —NO₂ and halogen;

R⁸ is hydrogen, (C₁-C₆)alkyl, aryl (C₁-C₆)alkyl, —C(O)R¹⁴ or —C(O)OR¹⁴;

R⁹ and R¹⁷ are independently 1-3 groups independently selected from the group consisting of hydrogen, (C₁-C₆)alkyl, (C₁-C₆)alkoxy, —C(O)OH, NO₂, —NR¹⁴R¹⁵, OH and halogeno;

R¹⁴ and R¹⁵ are independently selected from the group consisting of hydrogen, (C₁-C₆)alkyl; aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy.

Methods for making compounds of Formula IV are well known to those skilled in the art. Non-limiting examples of suitable methods are disclosed in U.S. Pat. No. 5,656,624, which is incorporated herein by reference.

In another embodiment, substituted azetidinones useful in the compositions, therapeutic combinations and methods of the present invention are represented by Formula (V): 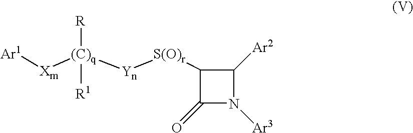
or a pharmaceutically acceptable salt thereof or a solvate thereof, or an ester thereof, wherein, in Formula (V) above:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(lower alkyl)₂—;

R is —OR⁶, —OC(O)R⁶, —OC(O)OR⁹ or —OC(O)NR⁶R⁷; R¹ is hydrogen, lower alkyl or aryl; or R and R¹ together are =0;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀C(O)NR⁶R⁷, -(lower alkylene)C(O)OR⁶ and —CH═CH—C(O)OR⁶;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR R⁷, S(O)₀₋₂R⁹, —O(CH₂)₁₋₁₀—C(O)OR⁶, —O(CH₂)₁₋₁₀C(O)NR⁶R⁷, —CF₃, —CN, —NO₂, halogen, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —N R⁶R⁷, —NR⁶C(O)R⁷, —N R⁶C(O)OR⁹, —NR⁶C(O)N R⁷R⁸, —NR⁶S(O)₂R⁹, —C(O)OR⁶, —C(O)NR⁶R⁷, —C(O)R⁶, —S(O)₂NR⁶R⁷, —S(O)₀₋₂R⁹, —O(CH₂)₁₋₁₀C(O)OR⁶, —O(CH₂)₁₋₁₀C(O)NR⁶R⁷, —CF₃, —CN, —NO₂ and halogen.

Methods for making compounds of Formula V are well known to those skilled in the art. Non-limiting examples of suitable methods are disclosed in U.S. Pat. No. 5,624,920, which is incorporated herein by reference.

In another embodiment, substituted azetidinones useful in the compositions, therapeutic combinations and methods of the present invention are represented by Formula (VI): 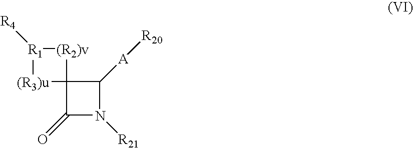
or a pharmaceutically acceptable salt thereof or a solvate thereof, or an ester thereof, wherein:

R¹ is:

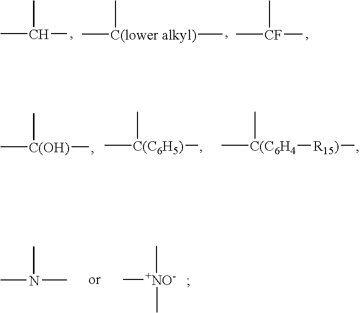

R² and R³ are independently selected from the group consisting of: —CH₂—, —CH(lower alkyl)-, —C(lower alkyl)₂—, —CH═CH— and —C(lower alkyl)=CH—; or

R¹ together with an adjacent R², or R¹ together with an adjacent R³, form a —CH═CH— or a —CH═C(lower alkyl)- group;

u and v are independently 0, 1, 2 or 3, provided both are not zero; provided that when R² is —CH═CH— or —C(lower alkyl)=CH—, v is 1; provided that when R³ is —CH═CH— or —C(lower alkyl)=CH—, u is 1; provided that when v is 2 or 3, each R² can be the same or different; and provided that when u is 2 or 3, each R³ can be the same or different;

R⁴ is selected from B—(CH₂)ₘC(O)—, wherein m is 0, 1, 2, 3, 4 or 5; B—(CH₂)_{q}—, wherein q is 0, 1, 2, 3, 4, 5 or 6; B—(CH₂)ₑ-Z-(CH₂)ᵣ—, wherein Z is —O—, —C(O)—, phenylene, —N(R⁸)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6; B-(C₂-C₆ alkenylene)-; B—(C₄-C₆ alkadienylene)-; B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6; B—(CH₂)_{f}—V—(CH₂)₉—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6; B—(CH₂)ₜ—V—(C₂-C₆ alkenylene)- or B—(C₂-C₆ alkenylene)-V—(CH₂)ₜ—, wherein V and t are as defined above, provided that the sum of t and the number of carbon atoms in the alkenylene chain is 2, 3, 4, 5 or 6;

B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is a C₃-C₆ cycloalkyl and s is 0, 1, 2, 3, 4, 5 or 6; or

R¹ and R⁴ together form the group

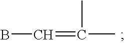

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

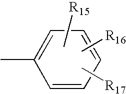

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R⁷-benzyl, benzyloxy, R⁷-benzyloxy, phenoxy, R⁷-phenoxy, dioxolanyl, NO₂, —N(R⁸)(R⁹), N(R⁸)(R⁹)-lower alkylene-, N(R⁸)(R⁹)-lower alkylenyloxy-, OH, halogeno, —CN, —N₃, —NHC(O)OR¹⁰, —NHC(O)R¹⁰, R¹¹(O)₂SNH—, (R¹¹(O)₂S)₂N—, —S(O)₂NH₂, —S(O)₀₋₂R⁸, tert-butyldimethyl-silyloxymethyl, —C(O)R¹², —C(O)OR¹⁹, —C(O)N(R⁸)(R⁹), —CH═CHC(O)R¹², -lower alkylene-C(O)R¹², R¹⁰C(O)(lower alkylenyloxy)-, N(R⁸)(R⁹)C(O)(lower alkylenyloxy)- and 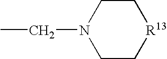
for substitution on ring carbon atoms, and the substituents on the substituted heteroaryl ring nitrogen atoms, when present, are selected from the group consisting of lower alkyl, lower alkoxy, —C(O)OR¹⁰, —C(O)R¹⁰, OH, N(R⁸)(R⁹)-lower alkylene-, N(R⁸)(R⁹)-lower alkylenyloxy-, —S(O)₂NH₂ and 2-(trimethylsilyl)-ethoxymethyl;

R⁷ is 1-3 groups independently selected from the group consisting of lower alkyl, lower alkoxy, —C(O)OH, NO₂, —N(R⁸)(R⁹), OH, and halogeno;

R⁸ and R⁹ are independently selected from H or lower alkyl;

R¹⁰ is selected from lower alkyl, phenyl, R⁷-phenyl, benzyl or R⁷-benzyl;

R¹¹ is selected from OH, lower alkyl, phenyl, benzyl, R⁷-phenyl or R⁷-benzyl;

R¹² is selected from H, OH, alkoxy, phenoxy, benzyloxy,

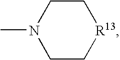

—N(R⁸)(R⁹), lower alkyl, phenyl or R⁷-phenyl;

R¹³ is selected from —O—, —CH₂—, —NH—, —N(lower alkyl)- or —NC(O)R¹⁹;

R¹⁵, R¹⁶ and R¹⁷ are independently selected from the group consisting of H and the groups defined for W; or R¹⁵ is hydrogen and R¹⁶ and R¹⁷, together with adjacent carbon atoms to which they are attached, form a dioxolanyl ring;

R¹⁹ is H, lower alkyl, phenyl or phenyl lower alkyl; and

R²⁰ and R²¹ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above.

Methods for making compounds of Formula VI are well known to those skilled in the art. Non-limiting examples of suitable methods are disclosed in U.S. Pat. No. 5,698,548, which is incorporated herein by reference.

In another embodiment, substituted azetidinones useful in the compositions, therapeutic combinations and methods of the present invention are represented by Formulas (VIIA) and (VIIB): 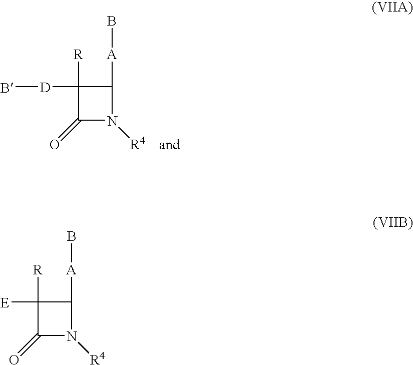
or a pharmaceutically acceptable salt, solvate, or ester thereof,
wherein:

A is —CH═CH—, —C≡C— or —(CH₂)ₚ— wherein p is 0, 1 or 2;

B is

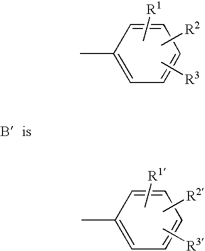

D is —(CH₂)ₘC(O)— or —(CH₂)_{q}— wherein m is 1, 2, 3 or 4 and q is 2, 3 or 4;

E is C₁₀ to C₂₀ alkyl or —C(O)—(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ—, wherein r is 0, 1, 2, or 3;

R¹, R², R³, R¹′, R²′, and R³′ are independently selected from the group consisting of hydrogen, lower alkyl, lower alkoxy, carboxy, NO₂, NH₂, OH, halogeno, lower alkylamino, dilower alkylamino, —NHC(O)OR⁵, R⁶(O)₂SNH— and —S(O)₂NH₂;

R⁴ is 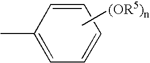
wherein n is 0, 1, 2 or 3;

R⁵ is lower alkyl; and

R⁶ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino; or a pharmaceutically acceptable salt thereof or a solvate thereof.

In another embodiment, sterol absorption inhibitors useful in the compositions and methods of the present invention are represented by Formula (VIII): 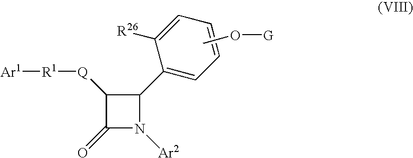
or a pharmaceutically acceptable salt thereof or a solvate thereof, or an ester thereof, wherein, in Formula (VIII) above,
R²⁶ is H or OG¹;
G and G′ are independently selected from the group consisting of

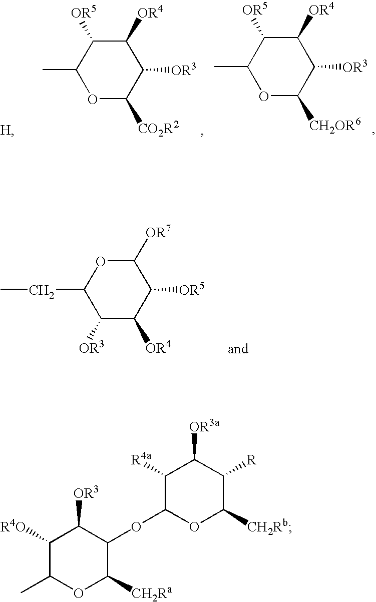

provided that when R² is H or OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group

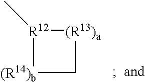

R¹ is selected from the group consisting of
—(CH₂)_{q}—, wherein q is 2-6, provided that when Q forms a spiro ring, q can also be zero or 1;
—(CH₂)ₑ-E-(CH₂)ᵣ, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;

R¹² is:

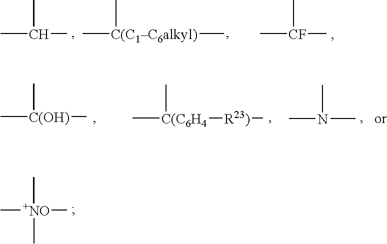

R¹³ and R¹⁴ are independently selected from the group consisting of

—CH₂—, —CH(C₁-C₆ alkyl)-, —C((C₁-C₆) alkyl)₂, —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero;

provided that when R¹³ is —CH═CH— or —C(C₁-C₆ alkyl)═CH—, a is 1;

provided that when R¹⁴ is —CH═CH— or —C(C₁-C₆ alkyl)=CH—, b is 1;

provided that when a is 2 or 3, each R¹³ can be the same or different; and

provided that when b is 2 or 3, each R¹⁴ can be the same or different;

and when Q is a bond, R¹ also can be:

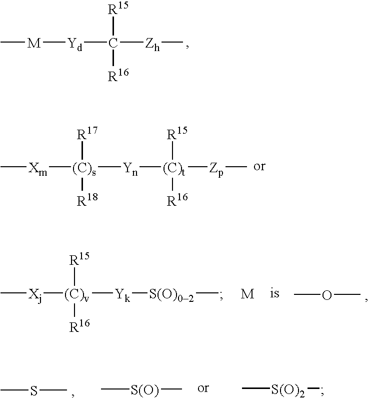

X, Y and Z are independently selected from the group consisting of —CH₂—, —CH(C₁-C₆)alkyl- and —C((C₁-C₆)alkyl)₂;

R¹⁰ and R¹¹ are independently selected from the group consisting of 1-3 substituents independently selected from the group consisting of (C₁-C₆)alkyl, —OR19, —OC(O)R¹⁹, —OC(O)OR²¹, —O(CH₂)₁₋₅OR¹⁹, —OC(O)NR¹⁹R²⁰, —NR¹⁹R²⁰, —NR₁₉C(O)R²⁰, —NR¹⁹C(O)OR¹⁹, —NR¹⁹C(O)NR²⁰R²⁵, —NR¹⁹S(O)₂R², —C(O)OR¹⁹, —C(O)NR¹⁹R²⁰, —C(O)R¹⁹, —S(O)₂NR¹⁹R²⁰, S(O)₀₋₂R²¹, —O(CH₂)₁₋₁₀—C(O)OR¹⁹, —O(CH₂)₁₋₁₀C(O)NR¹⁹R²⁰, —(C₁-C₆ alkylene)-C(O)OR¹⁹, —CH═CH—C(O)OR¹⁹, —CF₃, —CN, —NO₂ and halogen;

R¹⁵ and R¹⁷ are independently selected from the group consisting of —OR¹⁹, —OC(O)R¹⁹, —OC(O)OR²¹ and —OC(O)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

and when Q is a bond and R¹ is 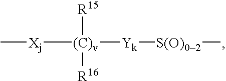
Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —C(O)OR¹⁹;

R²³ and R²⁴ are independently 1-3 groups independently selected from the group consisting of H, (C₁-C₆)alkyl, (C₁-C₆)alkoxy, —C(O)OH, NO₂, —NR¹⁹R²⁰, —OH and halogeno; and

R²⁵ is H, —OH or (C₁-C₆)alkoxy.

Methods for making compounds of Formula VIII are well known to those skilled in the art. Non-limiting examples of suitable methods are disclosed in U.S. Pat. No. 5,756,470, which is incorporated herein by reference.

In another embodiment, substituted azetidinones useful in the compositions and methods of the present invention are represented by Formula (IX) below: 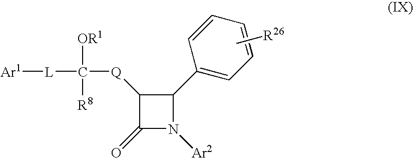
or a pharmaceutically acceptable salt, solvate, or ester thereof, wherein in Formula (IX):

R¹ is selected from the group consisting of H, G, G¹, G², —SO₃H and —PO₃H;
G is selected from the group consisting of: H,

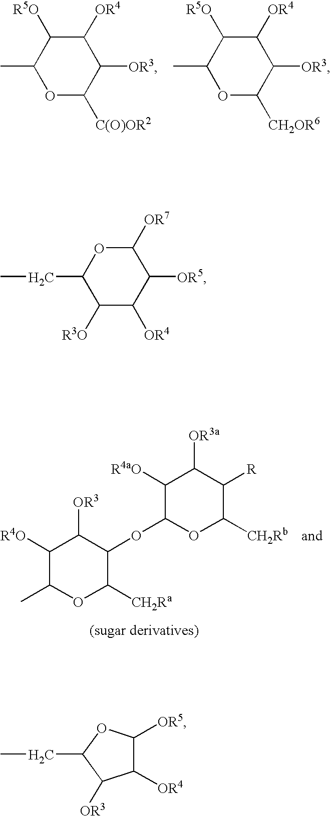

wherein R, R^{a} and R^{b} are each independently selected from the group consisting of H, —OH, halo, —NH₂, azido, (C₁-C₆)alkoxy(C₁-C₆)alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are each independently selected from the group consisting of H, (C₁-C₆)alkyl, acetyl, aryl and aryl(C₁-C₆)alkyl;

R³, R⁴, R⁵, R⁷, R^{3a} and R^{4a} are each independently selected from the group consisting of H, (C₁-C₆)alkyl, acetyl, aryl(C₁-C₆)alkyl, —C(O)(C₁-C₆)alkyl and —C(O)aryl;

R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents which are each independently selected from the group consisting of H, halo, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N(C₁-C₄)alkyl)₂, —C(O)—(C₁-C₄)alkyl, —C(O)—(C₁-C₄)alkoxy and pyrrolidinylcarbonyl; or R³² is a covalent bond and R³¹ the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

G¹ is represented by the structure: 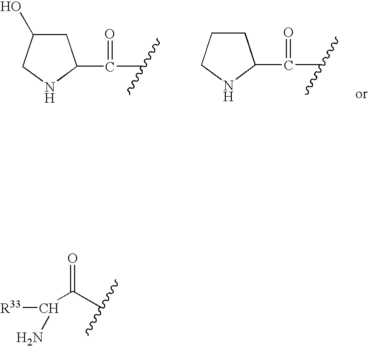
wherein R³³ is independently selected from the group consisting of unsubstituted alkyl, R³⁴-substituted alkyl, (R³⁵)(R³⁶)alkyl-,

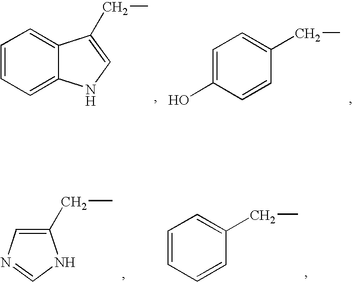

—R³⁴ is one to three substituents, each R³⁴ being independently selected from the group consisting of HO(O)C—, HO—, HS—, (CH₃)S—, H₂N—, (NH₂)(NH)C(NH)—, (NH₂)C(O)— and HO(O)CCH(NH₃⁺)CH₂SS—;

R³⁵ is independently selected from the group consisting of H and NH₂—;

R³⁶ is independently selected from the group consisting of H, unsubstituted alkyl, R³⁴-substituted alkyl, unsubstituted cycloalkyl and R³⁴-substituted cycloalkyl;

G² is represented by the structure: 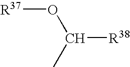
wherein R³⁷ and R³⁸ are each independently selected from the group consisting of (C₁-C₆)alkyl and aryl;

R²⁶ is one to five substituents, each R²⁶ being independently selected from the group consisting of:
a) H;
b) —OH;
c) —OCH₃;
d) fluorine;
e) chlorine;
f) —O-G;
g) —O-G¹;
h) —O-G²;
i) —SO₃H; and
j) —PO₃H;
   provided that when R¹ is H, R²⁶ is not H, —OH, —OCH₃ or —O-G;

Ar¹ is aryl, R¹⁰-substituted aryl, heteroaryl or R¹⁰-substituted heteroaryl;

Ar² is aryl, R¹¹-substituted aryl, heteroaryl or R¹¹-substituted heteroaryl;

L is selected from the group consisting of:

a) a covalent bond;

b) —(CH₂)_{q}—, wherein q is 1-6;

c) —(CH₂)ₑ-E-(CH₂)ᵣ—, wherein E is —O—, —C(O)—, phenylene, —NR²²— or
—S(O)₀₋₂—, e is 0-5 and r is 0-5, provided that the sum of e and r is 1-6;

d) —(C₂-C₆)alkenylene-;

e) —(CH₂)_{f}—V—(CH₂)_{g}—, wherein V is C₃-C₆cycloalkylene, f is 1-5 and g is 0-5, provided that the sum of f and g is 1-6; and

f) 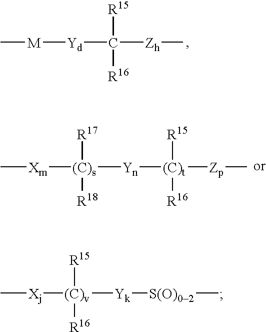
wherein M is —O—, —S—, —S(O)— or —S(O)₂—;

X, Y and Z are each independently selected from the group consisting of

—CH₂—, —CH(C₁-C₆)alkyl- and —C((C₁-C₆)alkyl)₂—;

R⁸ is selected from the group consisting of H and alkyl;
R¹⁰ and R¹¹ are each independently selected from the group consisting of 1-3 substituents which are each independently selected from the group consisting of (C₁-C₆)alkyl, —OR¹⁹, —OC(O)R¹⁹, —OC(O)OR²¹, —O(CH₂)₁₋₅OR¹⁹, —OC(O)NR¹⁹R²⁰, —NR¹⁹R²⁰, —NR¹⁹C(O)R², —NR¹⁹C(O)OR²¹, —NR¹⁹C(O)NR²OR²⁵, —N R¹⁹S(O)₂R², —C(O)OR¹⁹, —C(O)NR¹⁹R²⁰, —C(O)R¹⁹, —S(O)₂NR¹⁹R²⁰, S(O)₀₋₂R², —O(CH₂)₁₋₁₀—C(O)OR¹⁹, —O(CH₂)₁₋₁₀C(O)NR¹⁹R²⁰, —(C₁-C₆ alkylene)-C(O)OR¹⁹, —CH═CH—C(O)OR¹⁹, —CF₃, —CN, —NO₂ and halo;

R¹⁵ and R¹⁷ are each independently selected from the group consisting of —OR¹⁹, —OC(O)R¹⁹, —OC(O)OR²¹, —OC(O)NR¹⁹R²⁰;

R¹⁶ and R¹⁸ are each independently selected from the group consisting of H, (C₁-C₆)alkyl and aryl;

or R¹⁵ and R¹⁶ together are ═O, or R¹⁷ and R¹⁸ together are ═O;

d is 1, 2 or 3;

h is 0, 1, 2, 3 or 4;

s is 0 or 1;

t is 0 or 1;

m, n and p are each independently selected from 0-4;

provided that at least one of s and t is 1, and the sum of m, n, p, s and t is 1-6; provided that when p is 0 and t is 1, the sum of m, n and p is 1-5; and provided that when p is 0 and s is 1, the sum of m, t and n is 1-5;

v is 0 or 1;

j and k are each independently 1-5, provided that the sum of j, k and v is 1-5;

Q is a bond, —(CH₂)_{q}—, wherein q is 1-6, or, with the 3-position ring carbon of the azetidinone, forms the spiro group

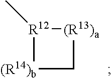

wherein R¹² is

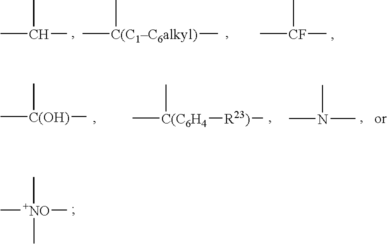

R¹³ and R¹⁴ are each independently selected from the group consisting of —CH₂—, —CH(C₁-C₆ alkyl)-, —C((C₁-C₆) alkyl)₂, —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;

a and b are each independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)═CH—, b is 1; provided that when a is 2 or 3, each R¹³ can be the same or different; and provided that when b is 2 or 3, each R¹⁴ can be the same or different;
and when Q is a bond and L is 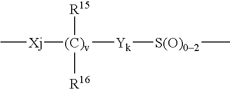
then Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are each independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —C(O)OR¹⁹;

R²³ and R²⁴ are each independently selected from the group consisting of 1-3 substituents which are each independently selected from the group consisting of H, (C₁-C₆)alkyl, (C₁-C₆)alkoxy, —C(O)OH, NO₂, —NR¹⁹R²⁰, —OH and halo; and

R²⁵ is H, —OH or (C₁-C₆)alkoxy.

Examples of compounds of Formula (IX) which are useful in the methods and combinations of the present invention and methods for making such compounds are disclosed in U.S. patent application Ser. No. 10/166,942, filed Jun. 11, 2002, incorporated herein by reference.

An example of a useful compound of this invention is one represented by the formula X: 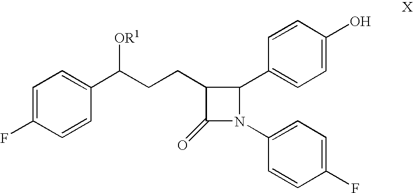
wherein R¹ is defined as above.

A more preferred compound is one represented by formula XI:

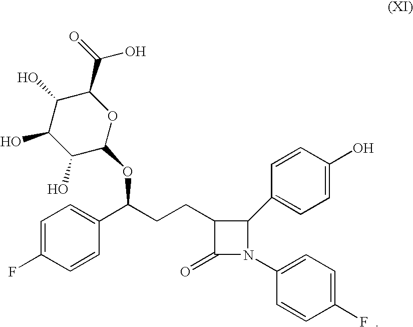

Another useful compound is represented by Formula XII:

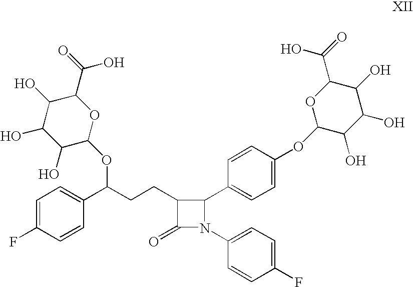

Other useful substituted azetidinone compounds include N-sulfonyl-2-azetidinones such as are disclosed in U.S. Pat. No. 4,983,597, ethyl 4-(2-oxoazetidin-4-yl)phenoxy-alkanoates such as are disclosed in Ram et al., Indian J. Chem. Sect. B. 29B, 12 (1990), p. 1134-7, diphenyl azetidinones and derivatives disclosed in U.S. Patent Publication Nos. 2002/0039774, 2002/0128252, 2002/0128253 and 2002/0137689, 2004/063929, WO 2002/066464, U.S. Pat. Nos. 6,498,156 and 6,703,386, each of which is incorporated by reference herein.

Other sterol absorption inhibitors useful in the compositions, therapeutic combinations and methods of the present invention are described in WO 2004/005247, WO 2004/000803, WO 2004/000804, WO 2004/000805, WO 0250027, U.S. published application 2002/0137689, and the compounds described in L. Kværnø et al., Angew. Chem. Int. Ed., 2004, vol. 43, pp. 4653-4656, all of which are incorporated herein by reference. An illustrative compound of Kværnø et al. is:

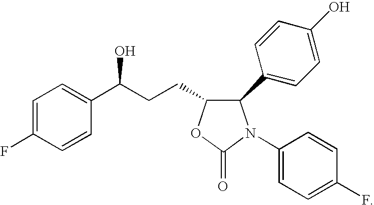

The compounds of Formulae I-XII can be prepared by known methods, including the methods discussed above and, for example, in WO 93/02048, U.S. Pat. Nos. 5,306,817 and 5,561,227, herein incorporated by reference, which describe the preparation of compounds wherein —R¹-Q- is alkylene, alkenylene or alkylene interrupted by a hetero atom, phenylene or cycloalkylene; WO 94/17038 and U.S. Pat. No. 5,698,548, herein incorporated by reference, describe the preparation of compounds wherein Q is a spirocyclic group; WO 95/08532, U.S. Pat. No. 5,631,365, U.S. Pat. No. 5,767,115, U.S. Pat. No. 5,846,966, and U.S. R.E. 37,721, herein incorporated by reference, describe the preparation of compounds wherein —R¹-Q- is a hydroxy-substituted alkylene group; PCT/US95/03196, herein incorporated by reference, describes compounds wherein —R¹-Q- is a hydroxy-substituted alkylene attached to the Ar¹ moiety through an —O— or S(O)₀₋₂— group; and U.S. Ser. No. 08/463,619, filed Jun. 5, 1995, herein incorporated by reference, describes the preparation of compounds wherein —R¹-Q- is a hydroxy-substituted alkylene group attached to the azetidinone ring by a —S(O)₀₋₂— group. Each of the above patents or publications are herein incorporated by reference in their entirety.

The daily dose of the sterol absorption inhibitor(s) administered to the subject can range from about 0.1 to about 1000 mg per day, preferably about 0.25 to about 50 mg/day, and more preferably about 10 mg per day, given in a single dose or 2-4 divided doses. The exact dose, however, is determined by the attending clinician and is dependent on the potency of the compound administered, the age, weight, condition and response of the patient.

For administration of pharmaceutically acceptable salts of the above compounds, the weights indicated above refer to the weight of the acid equivalent or the base equivalent of the therapeutic compound derived from the salt.

In one embodiment of the present invention, the compositions or therapeutic combinations can further comprise one or more pharmacological or therapeutic agents or drugs such as cholesterol biosynthesis inhibitors and/or lipid-lowering agents discussed below.

In another embodiment, the composition or treatment can further comprise one or more cholesterol biosynthesis inhibitors coadministered with or in combination with the selective CB₁ receptor antagonist and substituted azetidinone or substituted β-lactam discussed above.

Generally, a total daily dosage of cholesterol biosynthesis inhibitor(s) can range from about 0.1 to about 160 mg per day, and preferably about 0.2 to about 80 mg/day in single or 2-3 divided doses.

In another embodiment, the composition or treatment comprises the compound of Formula (II) in combination with one or more selective CB₁ receptor antagonists and one or more cholesterol biosynthesis inhibitors. In this embodiment, preferably the selective CB₁ receptor antagonist is one of the compounds described in U.S. Pat. No. 5,624,941, herein incorporated by reference, such as for example, rimonabant. Preferably the cholesterol biosynthesis inhibitor comprises one or more HMG CoA reductase inhibitors, such as, for example, lovastatin, pravastatin and/or simvastatin. More preferably, the composition or treatment comprises rimonabant and the compound of Formula (II) in combination with simvastatin and ETC-216.

In another alternative embodiment, the compositions, therapeutic combinations or methods of the present invention can further comprise one or more bile acid sequestrants (insoluble anion exchange resins), coadministered with or in combination with selective CB₁ receptor antagonist(s) and substituted azetidinone or substituted β-lactam discussed above.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors that bind LDL from plasma to further reduce cholesterol levels in the blood.

Generally, a total daily dosage of bile acid sequestrant(s) can range from about 1 to about 50 grams per day, and preferably about 2 to about 16 grams per day in single or 2-4 divided doses.

In an alternative embodiment, the compositions or treatments of the present invention can further comprise one or more IBAT inhibitors. The IBAT inhibitors can inhibit bile acid transport to reduce LDL cholesterol levels. Generally, a total daily dosage of IBAT inhibitor(s) can range from about 0.01 to about 1000 mg/day, and preferably about 0.1 to about 50 mg/day in single or 2-4 divided doses.

In another alternative embodiment, the compositions or treatments of the present invention can further comprise nicotinic acid (niacin) and/or derivatives thereof. Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

Generally, a total daily dosage of nicotinic acid or a derivative thereof can range from about 500 to about 10,000 mg/day, preferably about 1000 to about 8000 mg/day, and more preferably about 3000 to about 6000 mg/day in single or divided doses.

In another alternative embodiment, the compositions or treatments of the present invention can further comprise one or more AcylCoA:Cholesterol O-acyltransferase (“ACAT”) Inhibitors, which can reduce LDL and VLDL levels. ACAT is an enzyme responsible for esterifying excess intracellular cholesterol and may reduce the synthesis of VLDL, which is a product of cholesterol esterification, and overproduction of apo B-100-containing lipoproteins. Generally, a total daily dosage of ACAT inhibitor(s) can range from about 0.1 to about 1000 mg/day in single or 2-4 divided doses.

In another alternative embodiment, the compositions or treatments of the present invention can further comprise one or more Cholesteryl Ester Transfer Protein (“CETP”) Inhibitors. CETP is responsible for the exchange or transfer of cholesteryl ester carrying HDL and triglycerides in VLDL. Pancreatic cholesteryl ester hydrolase (PCEH) inhibitors such as WAY-121898 also can be coadministered with or in combination.

Generally, a total daily dosage of CETP inhibitor(s) can range from about 0.01 to about 1000 mg/day, and preferably about 0.5 to about 20 mg/kg body weight/day in single or divided doses.

In another alternative embodiment, the compositions or treatments of the present invention can further comprise probucol or derivatives thereof, which can reduce LDL levels.

Generally, a total daily dosage of probucol or derivatives thereof can range from about 10 to about 2000 mg/day, and preferably about 500 to about 1500 mg/day in single or 2-4 divided doses.

In another alternative embodiment, the compositions or treatments of the present invention can further comprise low-density lipoprotein (LDL) receptor activators.

Generally, a total daily dosage of LDL receptor activator(s) can range from about 1 to about 1000 mg/day in single or 2-4 divided doses.

In another alternative embodiment, the compositions or treatments of the present invention can further comprise fish oil. Generally, a total daily dosage of fish oil or Omega 3 fatty acids can range from about 1 to about 30 grams per day in single or 2-4 divided doses.

In another alternative embodiment, the compositions or treatments of the present invention can further comprise natural water soluble fibers, such as psyllium, guar, oat and pectin, which can reduce cholesterol levels. Generally, a total daily dosage of natural water soluble fibers can range from about 0.1 to about 10 grams per day in single or 2-4 divided doses.

In another alternative embodiment, the compositions or treatments of the present invention can further comprise plant sterols, plant stanols and/or fatty acid esters of plant stanols, such as sitostanol ester used in BENECOL® margarine, which can reduce cholesterol levels. Generally, a total daily dosage of plant sterols, plant stanols and/or fatty acid esters of plant stanols can range from about 0.5 to about 20 grams per day in single or 2-4 divided doses.

In another alternative embodiment, the compositions or treatments of the present invention can further comprise antioxidants, such as probucol, tocopherol, ascorbic acid, β-carotene and selenium, or vitamins such as vitamin B₆ or vitamin B₁₂. Generally, a total daily dosage of antioxidants or vitamins can range from about 0.05 to about 10 grams per day in single or 2-4 divided doses.

In another alternative embodiment, the compositions or treatments of the present invention can further comprise monocyte and macrophage inhibitors such as polyunsaturated fatty acids (PUFA), thyroid hormones including throxine analogues such as CGS-26214 (a thyroxine compound with a fluorinated ring), gene therapy and use of recombinant proteins such as recombinant apo E. Generally, a total daily dosage of these agents can range from about 0.01 to about 1000 mg/day in single or 2-4 divided doses.

Also useful with the present invention are compositions or therapeutic combinations that further comprise hormone replacement agents and compositions. Useful hormone agents and compositions for hormone replacement therapy of the present invention include androgens, estrogens, progestins, their pharmaceutically acceptable salts and derivatives thereof. Combinations of these agents and compositions are also useful.

The dosage of androgen and estrogen combinations vary, desirably from about 1 mg to about 4 mg androgen and from about 1 mg to about 3 mg estrogen. Examples include, but are not limited to, androgen and estrogen combinations such as the combination of esterified estrogens (sodium estrone sulfate and sodium equilin sulfate) and methyltestosterone (17-hydroxy-17-methyl-, (17B)-androst-4-en-3-one) available from Solvay Pharmaceuticals, Inc., Marietta, Ga., under the tradename Estratest.

Estrogens and estrogen combinations may vary in dosage from about 0.01 mg up to 8 mg, desirably from about 0.3 mg to about 3.0 mg. Examples of useful estrogens and estrogen combinations include:

(a) the blend of nine (9) synthetic estrogenic substances including sodium estrone sulfate, sodium equilin sulfate, sodium 17 α-dihydroequilin sulfate, sodium 17 α-estradiol sulfate, sodium 17 β-dihydroequilin sulfate, sodium 17 α-dihydroequilenin sulfate, sodium 17 β-dihydroequilenin sulfate, sodium equilenin sulfate and sodium 17 β-estradiol sulfate; available from Duramed Pharmaceuticals, Inc., Cincinnati, Ohio, under the tradename Cenestin;

(b) ethinyl estradiol (19-nor-17 α-pregna-1,3,5(10)-trien-20-yne-3,17-diol; available by Schering Plough Corporation, Kenilworth, N.J., under the tradename Estinyl;

(c) esterified estrogen combinations such as sodium estrone sulfate and sodium equilin sulfate; available from Solvay under the tradename Estratab and from Monarch Pharmaceuticals, Bristol, Tenn., under the tradename Menest;

(d) estropipate (piperazine estra-1,3,5(10)-trien-17-one, 3-(sulfooxy)-estrone sulfate); available from Pharmacia & Upjohn, Peapack, N.J., under the tradename Ogen and from Women First Health Care, Inc., San Diego, Calif., under the tradename Ortho-Est; and

(e) conjugated estrogens (17 α-dihydroequilin, 17 α-estradiol, and 17 β-dihydroequilin); available from Wyeth-Ayerst Pharmaceuticals, Philadelphia, Pa., under the tradename Premarin.

Progestins and estrogens may also be administered with a variety of dosages, generally from about 0.05 to about 2.0 mg progestin and about 0.001 mg to about 2 mg estrogen, desirably from about 0.1 mg to about 1 mg progestin and about 0.01 mg to about 0.5 mg estrogen. Examples of progestin and estrogen combinations that may vary in dosage and regimen include:

(a) the combination of estradiol (estra-1, 3, 5 (10)-triene-3, 17 β-diol hemihydrate) and norethindrone (17 β-acetoxy-19-nor-17 α-pregn-4-en-20-yn-3-one); which is available from Pharmacia & Upjohn, Peapack, N.J., under the tradename Activella;

(b) the combination of levonorgestrel (d(−)-13 β-ethyl-17 α-ethinyl-17 β-hydroxygon-4-en-3-one) and ethinyl estradial; available from Wyeth-Ayerst under the tradename Alesse, from Watson Laboratories, Inc., Corona, Calif., under the tradenames Levora and Trivora, Monarch Pharmaceuticals, under the tradename Nordette, and from Wyeth-Ayerst under the tradename Triphasil;

(c) the combination of ethynodiol diacetate (19-nor-17 α-pregn-4-en-20-yne-3 β, 17-diol diacetate) and ethinyl estradiol; available from G.D. Searle & Co., Chicago, Ill., under the tradename Demulen and from Watson under the tradename Zovia;

(d) the combination of desogestrel (13-ethyl-11-methylene-18,19-dinor-17 α-pregn-4-en-20-yn-17-ol) and ethinyl estradiol; available from Organon under the tradenames Desogen and Mircette, and from Ortho-McNeil Pharmaceutical, Raritan, N.J., under the tradename Ortho-Cept;

(e) the combination of norethindrone and ethinyl estradiol; available from Parke-Davis, Morris Plains, N.J., under the tradenames Estrostep and femhrt, from Watson under the tradenames Microgestin, Necon, and Tri-Norinyl, from Ortho-McNeil under the tradenames Mod icon and Ortho-Novum, and from Warner Chilcott Laboratories, Rockaway, N.J., under the tradename Ovcon;

(f) the combination of norgestrel ((±)-13-ethyl-17-hydroxy-18, 19-dinor-17 α-preg-4-en-20-yn-3-one) and ethinyl estradiol; available from Wyeth-Ayerst under the tradenames Ovral and Lo/Ovral, and from Watson under the tradenames Ogestrel and Low-Ogestrel;

(g) the combination of norethindrone, ethinyl estradiol, and mestranol (3-methoxy-19-nor-17 α-pregna-1,3,5(10)-trien-20-yn-17-ol); available from Watson under the tradenames Brevicon and Norinyl;

(h) the combination of 17 β-estradiol (estra-1,3,5(10)-triene-3,17 β-diol) and micronized norgestimate (17 α-17-(Acetyloxyl)-13-ethyl-18,19-dinorpregn-4-en-20-yn-3-one3-oxime); available from Ortho-McNeil under the tradename Ortho-Prefest;

(i) the combination of norgestimate (18,19-dinor-17-pregn-4-en-20-yn-3-one, 17-(acetyloxy)-13-ethyl-, oxime, (17(α)-(+)-) and ethinyl estradiol; available from Ortho-McNeil under the tradenames Ortho Cyclen and Ortho Tri-Cyclen; and

(j) the combination of conjugated estrogens (sodium estrone sulfate and sodium equilin sulfate) and medroxyprogesterone acetate (20dione, 17-(acetyloxy)-6-methyl-, (6(α))-pregn-4-ene-3); available from Wyeth-Ayerst under the tradenames Premphase and Prempro.

In general, a dosage of progestins may vary from about 0.05 mg to about 10 mg or up to about 200 mg if microsized progesterone is administered. Examples of progestins include norethindrone; available from ESI Lederle, Inc., Philadelphia, Pa., under the tradename Aygestin, from Ortho-McNeil under the tradename Micronor, and from Watson under the tradename Nor-QD; norgestrel; available from Wyeth-Ayerst under the tradename Ovrette; micronized progesterone (pregn-4-ene-3,20-dione); available from Solvay under the tradename Prometrium; and medroxyprogesterone acetate; available from Pharmacia & Upjohn under the tradename Provera.

The compositions, therapeutic combinations or methods of the present invention can further comprise one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); alpha-blocking agents; kainite or AMPA receptor antagonists; leptin-lipolysis stimulated receptors; phosphodiesterase enzyme inhibitors; compounds having nucleotide sequences of the mahogany gene; fibroblast growth factor-10 polypeptides; monoamine oxidase inhibitors (such as befloxatone, moclobemide, brofaromine, phenoxathine, esuprone, befol, toloxatone, pirlindol, amiflamine, sercloremine, bazinaprine, lazabemide, milacemide and caroxazone); compounds for increasing lipid metabolism (such as evodiamine compounds); and lipase inhibitors (such as orlistat). Generally, a total dosage of the above-described obesity control medications can range from 1 to 3,000 mg/day, desirably from about 1 to 1,000 mg/day and more desirably from about 1 to 200 mg/day in single or 2-4 divided doses.

The compositions, therapeutic combinations or methods of the present invention can further comprise one or more blood modifiers which are chemically different from the substituted azetidinone and substituted P-lactam compounds (such as compounds I-XII above) and the lipid modulating agents discussed above, for example, they contain one or more different atoms, have a different arrangement of atoms or a different number of one or more atoms than the sterol absorption inhibitor(s) or lipid modulating agents discussed above. Useful blood modifiers include but are not limited to anti-coagulants (argatroban, bivalirudin, dalteparin sodium, desirudin, dicumarol, lyapolate sodium, nafamostat mesylate, phenprocoumon, tinzaparin sodium, warfarin sodium); antithrombotic (anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab, zolimomab aritox); fibrinogen receptor antagonists (roxifiban acetate, fradafiban, orbofiban, lotrafiban hydrochloride, tirofiban, xemilofiban, monoclonal antibody 7E3, sibrafiban); platelet inhibitors (cilostazol, clopidogrel bisulfate, epoprostenol, epoprostenol sodium, ticlopidine hydrochloride, aspirin, ibuprofen, naproxen, sulindae, idomethacin, mefenamate, droxicam, diclofenac, sulfinpyrazone, piroxicam, dipyridamole); platelet aggregation inhibitors (acadesine, beraprost, beraprost sodium, ciprostene calcium, itazigrel, lifarizine, lotrafiban hydrochloride, orbofiban acetate, oxagrelate, fradafiban, orbofiban, tirofiban, xemilofiban); hemorrheologic agents (pentoxifylline); lipoprotein associated coagulation inhibitors; Factor VIla inhibitors (4H-31-benzoxazin-4-ones, 4H-3,1-benzoxazin-4-thiones, quinazolin-4-ones, quinazolin-4-thiones, benzothiazin-4-ones, imidazolyl-boronic acid-derived peptide analogues TFPI-derived peptides, naphthalene-2-sulfonic acid {1-[3-(aminoiminomethyl)-benzyl]-2-oxo-pyrrolidin-3-(S)-yl} amide trifluoroacetate, dibenzofuran-2-sulfonic acid {1-[3-(aminomethyl)-benzyl]-5-oxo-pyrrolidin-3-yl}-amide, tolulene-4-sulfonic acid {1-[3-(aminoiminomethyl)-benzyl]-2-oxo-pyrrolidin-3-(S)-yl}-amide trifluoroacetate, 3,4-dihydro-1H-isoquinoline-2-sulfonic acid {1-[3-(aminoiminomethyl)-benzyl]-2-oxo-pyrrolin-3-(S)-yl}-amide trifluoroacetate); Factor Xa inhibitors (disubstituted pyrazolines, disubstituted triazolines, substituted n-[(aminoiminomethyl)phenyl] propylamides, substituted n-[(aminomethyl)phenyl] propylamides, tissue factor pathway inhibitor (TFPI), low molecular weight heparins, heparinoids, benzimidazolines, benzoxazolinones, benzopiperazinones, indanones, dibasic (amidinoaryl) propanoic acid derivatives, amidinophenyl-pyrrolidines, amidinophenyl-pyrrolines, amidinophenyl-isoxazolidines, amidinoindoles, amidinoazoles, bis-arlysulfonylaminobenzamide derivatives, peptidic Factor Xa inhibitors).

The compositions, therapeutic combinations or methods of the present invention can further comprise one or more cardiovascular agents which are chemically different from the substituted azetidinone and substituted β-lactam compounds (such as compounds I-XI above) and the lipid modulating agents discussed above, for example, they contain one or more different atoms, have a different arrangement of atoms or a different number of one or more atoms than the sterol absorption inhibitor(s) or PPAR receptor activators discussed above. Useful cardiovascular agents include but are not limited to calcium channel blockers (clentiazem maleate, amlodipine besylate, isradipine, nimodipine, felodipine, nilvadipine, nifedipine, teludipine hydrochloride, diltiazem hydrochloride, belfosdil, verapamil hydrochloride, fostedil); adrenergic blockers (fenspiride hydrochloride, labetalol hydrochloride, proroxan, alfuzosin hydrochloride, acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate, nebivolol); adrenergic stimulants; angiotensin converting enzyme (ACE) inhibitors (benazepril hydrochloride, benazeprilat, captopril, delapril hydrochloride, fosinopril sodium, libenzapril, moexipril hydrochloride, pentopril, perindopril, quinapril hydrochloride, quinaprilat, ramipril, spirapril hydrochloride, spiraprilat, teprotide, enalapril maleate, lisinopril, zofenopril calcium, perindopril erbumine); antihypertensive agents (althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium, guanfacine hydrochloride, methyldopa, metoprolol succinate, moexipril hydrochloride, monatepil maleate, pelanserin hydrochloride, phenoxybenzamine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate, bevantolol hydrochloride); angiotensin II receptor antagonists (candesartan, irbesartan, losartan potassium, candesartan cilexetil, telmisartan); anti-anginal agents (amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochoride, tosifen, verapamil hydrochloride); coronary vasodilators (fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexiline maleate, prenylamine, propatyl nitrate, terodiline hydrochloride, tolamolol, verapamil); diuretics (the combination product of hydrochlorothiazide and spironolactone and the combination product of hydrochlorothiazide and triamterene).

The compositions, therapeutic combinations or methods of the present invention can further comprise one or more antidiabetic medications for reducing blood glucose levels in a human. Useful antidiabetic medications include, but are not limited to, drugs that reduce energy intake or suppress appetite, drugs that increase energy expenditure and nutrient-partitioning agents. Suitable antidiabetic medications include, but are not limited to, sulfonylurea (such as acetohexamide, chlorpropamide, gliamilide, gliclazide, glimepiride, glipizide, glyburide, glibenclamide, tolazamide, and tolbutamide), meglitinide (such as repaglinide and nateglinide), biguanide (such as metformin and buformin), alpha-glucosidase inhibitor (such as acarbose, miglitol, camiglibose, and voglibose), certain peptides (such as amlintide, pramlintide, exendin, and GLP-1 agonistic peptides), and orally administrable insulin or insulin composition for intestinal delivery thereof. Generally, a total dosage of the above-described antidiabetic medications can range from 0.1 to 1,000 mg/day in single or 2-4 divided doses.

Mixtures of any of the pharmacological or therapeutic agents described above can be used in the compositions and therapeutic combinations of the present invention.

The compositions and therapeutic combinations of the present invention can be administered to a subject or mammal in need of such treatment in a therapeutically effective amount to treat one or more conditions, for example vascular conditions such as atherosclerosis, hyperlipidaemia (including but not limited to hypercholesterolemia, hypertriglyceridaemia, sitosterolemia), vascular inflammation, stroke, diabetes, metabolic syndrome, obesity, and/or reduce the level of sterol(s) in the plasma. The compositions and treatments can be administered by any suitable means which produce contact of these compounds with the site of action in the body, for example in the plasma, liver or small intestine of a mammal or human.

The pharmaceutical treatment compositions and therapeutic combinations of the present invention can further comprise one or more pharmaceutically acceptable carriers, one or more excipients and/or one or more additives. Non-limiting examples of pharmaceutically acceptable carriers include solids and/or liquids such as ethanol, glycerol, water and the like. The amount of carrier in the treatment composition can range from about 5 to about 99 weight percent of the total weight of the treatment composition or therapeutic combination. Non-limiting examples of suitable pharmaceutically acceptable excipients and additives include non-toxic compatible fillers, binders such as starch, disintegrants, buffers, preservatives, anti-oxidants, lubricants, flavorings, thickeners, coloring agents, emulsifiers and the like. The amount of excipient or additive can range from about 0.1 to about 90 weight percent of the total weight of the treatment composition or therapeutic combination. One skilled in the art would understand that the amount of carrier(s), excipients and additives (if present) can vary.

The treatment compositions of the present invention can be administered in any conventional dosage form, preferably an oral dosage form such as a capsule, tablet, powder, cachet, suspension or solution. The formulations and pharmaceutical compositions can be prepared using conventional pharmaceutically acceptable and conventional techniques.

It is contemplated that where the two active ingredients are administered as a single composition, the dosage forms disclosed above for substituted azetidinone or β-lactam compounds may readily be modified using the knowledge of one skilled in the art.

Since the present invention relates to treating conditions as discussed above, such as reducing the plasma sterol (especially cholesterol) concentrations or levels by treatment with a combination of active ingredients wherein the active ingredients may be administered separately, the invention also relates to combining separate pharmaceutical compositions in kit form. That is, a kit is contemplated wherein two separate units are combined: a pharmaceutical composition comprising at least one selective CB₁ receptor antagonist and a separate pharmaceutical composition comprising at least one cholesterol lowering compound as described above. The kit will preferably include directions for the administration of the separate components. The kit form is particularly advantageous when the separate components must be administered in different dosage forms (e.g., oral and parenteral) or are administered at different dosage intervals.

The treatment compositions and therapeutic combinations of the present invention can inhibit the intestinal absorption of cholesterol in mammals, as shown in the Example below, and can be useful in the treatment and/or prevention of conditions, for example vascular conditions, such as atherosclerosis, hypercholesterolemia and sitosterolemia, stroke, obesity and lowering of plasma levels of cholesterol in mammals, in particular in mammals.

In another embodiment of the present invention, the compositions and therapeutic combinations of the present invention can inhibit sterol or 5α-stanol absorption or reduce plasma concentration of at least one sterol selected from the group consisting of phytosterols (such as sitosterol, campesterol, stigmasterol and avenosterol) and/or 5α-stanol (such as cholestanol, 5α-campestanol, 5α-sitostanol), cholesterol and mixtures thereof. The plasma concentration can be reduced by administering to a mammal in need of such treatment an effective amount of at least one treatment composition or therapeutic combination comprising at least one selective CB₁ receptor antagonist and at least one cholesterol lowering compound, for example a sterol absorption inhibitor described above. The reduction in plasma concentration of sterols or 5α-stanols can range from about 1 to about 70 percent, and preferably about 10 to about 50 percent. Methods of measuring serum total blood cholesterol and total LDL cholesterol are well known to those skilled in the art and for example include those disclosed in PCT WO 99/38498 at page 11, incorporated by reference herein. Methods of determining levels of other sterols in serum are disclosed in H. Gylling et al., “Serum Sterols During Stanol Ester Feeding in a Mildly Hypercholesterolemic Population”, J. Lipid Res. 40: 593-600 (1999), incorporated by reference herein.

The treatments of the present invention can also reduce the size or presence of plaque deposits in vascular vessels. The plaque volume can be measured using (IVUS), in which a tiny ultrasound probe is inserted into an artery to directly image and measure the size of atherosclerotic plaques, in a manner well know to those skilled in the art.

Illustrating the invention are the following examples that, however, are not to be considered as limiting the invention to their details. Unless otherwise indicated, all parts and percentages in the following examples, as well as throughout the specification, are by weight.

### EXAMPLES

### Preparation of Compound of Formula (II)

Step 1): To a solution of (S)-4-phenyl-2-oxazolidinone (41 g, 0.25 mol) in CH₂Cl₂ (200 mL), was added 4-dimethylaminopyridine (2.5 g, 0.02 mol) and triethylamine (84.7 mL, 0.61 mol) and the reaction mixture was cooled to 0° C. Methyl-4-(chloroformyl)butyrate (50 g, 0.3 mol) was added as a solution in CH₂Cl₂ (375 mL) dropwise over 1 h, and the reaction was allowed to warm to 22° C. After 17 h, water and H₂SO₄ (2N, 100 mL), was added the layers were separated, and the organic layer was washed sequentially with NaOH (10%), NaCl (sat'd) and water. The organic layer was dried over MgSO₄ and concentrated to obtain a semicrystalline product.

Step 2): To a solution of TiCl₄ (18.2 mL, 0.165 mol) in CH₂Cl₂ (600 mL) at 0° C., was added titanium isopropoxide (16.5 mL, 0.055 mol). After 15 min, the product of Step 1 (49.0 g, 0.17 mol) was added as a solution in CH₂Cl₂ (100 mL). After 5 min., diisopropylethylamine (DIPEA) (65.2 mL, 0.37 mol) was added and the reaction mixture was stirred at 0° C. for 1 h, the reaction mixture was cooled to −20° C., and 4-benzyloxybenzylidine(4-fluoro)aniline (114.3 g, 0.37 mol) was added as a solid. The reaction mixture was stirred vigorously for 4 h at −20° C., then acetic acid was added as a solution in CH₂Cl₂ dropwise over 15 min, the reaction mixture was allowed to warm to 0° C., and H₂SO₄ (2N) was added. The reaction mixture was stirred an additional 1 h, the layers were separated, washed with water, separated and the organic layer was dried. The crude product was crystallized from ethanol/water to obtain the pure intermediate.

Step 3): To a solution of the product of Step 2 (8.9 g, 14.9 mmol) in toluene (100 mL) at 50° C., was added N,O-bis(trimethylsilyl)acetamide (BSA) (7.50 mL, 30.3 mmol). After 0.5 h, solid TBAF (0.39 g, 1.5 mmol) was added and the reaction mixture stirred at 50° C. for an additional 3 h. The reaction mixture was cooled to 22° C., CH₃OH (10 mL), was added. The reaction mixture was washed with HCl (1 N), NaHCO₃ (1 N) and NaCl (sat'd.), and the organic layer was dried over MgSO₄.

Step 4): To a solution of the product of Step 3 (0.94 g, 2.2 mmol) in CH₃OH (3 mL), was added water (1 mL) and LiOH.H₂O (102 mg, 2.4 mmole). The reaction mixture was stirred at 22° C. for 1 h and then additional LiOH.H₂O (54 mg, 1.3 mmole) was added. After a total of 2 h, HCl (1 N) and EtOAc was added, the layers were separated, the organic layer was dried and concentrated in vacuo. To a solution of the resultant product (0.91 g, 2.2 mmol) in CH₂Cl₂ at 22° C., was added ClCOCOCl (0.29 mL, 3.3 mmol) and the mixture stirred for 16 h. The solvent was removed in vacuo.

Step 5): To an efficiently stirred suspension of 4-fluorophenylzinc chloride (4.4 mmol) prepared from 4-fluorophenylmagnesium bromide (1 M in THF, 4.4 mL, 4.4 mmol) and ZnCl₂ (0.6 g, 4.4 mmol) at 4° C., was added tetrakis(triphenyl-phosphine)palladium (0.25 g, 0.21 mmol) followed by the product of Step 4 (0.94 g, 2.2 mmol) as a solution in THF (2 mL). The reaction was stirred for 1 h at 0° C. and then for 0.5 h at 22° C. HCl (1 N, 5 mL) was added and the mixture was extracted with EtOAc. The organic layer was concentrated to an oil and purified by silica gel chromatography to obtain 1-(4-fluorophenyl)-4(S)-(4-hydroxyphenyl)-3(R)-(3-oxo-3-phenylpropyl)-2-azetidinone:

HRMS calc'd for C₂₄H₁₉F₂NO₃=408.1429, found 408.1411.

Step 6): To the product of Step 5 (0.95 g, 1.91 mmol) in THF (3 mL), was added (R)-tetrahydro-1-methyl-3,3-diphenyl-1H,3H-pyrrolo-[1,2-c][1,3,2] oxazaborole (120 mg, 0.43 mmol) and the mixture was cooled to −20° C. After 5 min, borohydride-dimethylsulfide complex (2M in THF, 0.85 mL, 1.7 mmol) was added dropwise over 0.5 h. After a total of 1.5 h, CH₃OH was added followed by HCl (1 N) and the reaction mixture was extracted with EtOAc to obtain 1-(4-fluorophenyl)-3(R)-[3(S)-(4-fluorophenyl)-3-hydroxypropyl)]-4(S)-[4-(phenylmethoxy)phenyl]-2-azetidinone (compound 6A-1) as an oil. ¹H in CDCl₃ d H3=4.68. J=2.3 Hz. Cl (M+H) 500.

Use of (S)-tetra-hydro-1-methyl-3,3-diphenyl-1H,3H-pyrrolo-[1,2-c][1,3,2] oxazaborole gives the corresponding 3(R)-hydroxypropyl azetidinone (compound 6B-1). ¹H in CDCl₃ d H3=4.69. J=2.3 Hz. Cl (M+H) 500.

To a solution of compound 6A-1 (0.4 g, 0.8 mmol) in ethanol (2 mL), was added 10% Pd/C (0.03 g) and the reaction mixture was stirred under a pressure (60 psi) of H₂ gas for 16 h. The reaction mixture was filtered and the solvent was concentrated to obtain compound 6A. Mp 164-166° C.; Cl (M+H) 410. [α]_{D}²⁵=28.1° (c 3, CH₃H). Elemental analysis calc'd for C₂₄H₂₁F₂NO₃: C, 70.41; H, 5.17; N, 3.42; found C, 70.25; H, 5.19; N, 3.54.

Similarly treat compound 6B-1 to obtain compound 6B. Mp 129.5-132.5° C.; Cl (M+H) 410. Elemental analysis calc'd for C₂₄H₂₁F2NO₃: C, 70.41; H, 5.17; N, 3.42; found C, 70.30; H, 5.14; N, 3.52.

Step 6′ (Alternative): To a solution of the product of Step 5 (0.14 g, 0.3 mmol) in ethanol (2 mL), was added 10% Pd/C (0.03 g) and the reaction was stirred under a pressure (60 psi) of H₂ gas for 16 h. The reaction mixture was filtered and the solvent was concentrated to afford a 1:1 mixture of compounds 6A and 6B.

Method for Measuring CB₁ and CB₂ Receptor Affinity

Materials:

Buffer: 50 mM Tris, HCl, pH 7.4+5 mM MgCl2+2.5 mM EDTA+0.1% BSA (1 mg/mL)

Ligand: ³H—CP55,940-168 Ci/mmol−1 μCi/μL−volume of label in assay=180 μL.

For saturation studies, prepare a starting concentration of 5 nM of the ³H—CP55,940 ligand by adding 6 μL of 3H-CP55,940 per 3.2 mL (0.336 μCi/180 μL) of buffer for a dpm of ˜750,000 dpm/180 μL. Dilute this solution 1:2 for a total of 10 concentrations.

For competition studies, prepare a final conc. of 0.75 nM by adding 6 μL of ³H-CP55,940 ligand per 20 mL (0.05 μCi/180 μL) to yield a final dpm of ˜100,000 dpm/180 μL.

Selective CB₁ receptor antagonist compound solutions: Dilute 10 mM stock concentrations of selective CB₁ receptor antagonist in 100% DMSO 1:1667 in 100% DMSO, to yield 60 μM selective CB₁ receptor antagonist in 100% DMSO (10 μL drug+1657 μL DMSO). Dilute these in half log steps in 100% DMSO using, for example, a Tecan Genesis robot. 20 μL additions of the selective CB₁ receptor antagonist in 100% DMSO into the assay volume of 400 μL provides a final concentration of 3 μM in 5% DMSO, which after dilution will give final concentrations of 0.0001 μM-3 μM.

Non-specific: For both CB₁ and CB₂ assays, use 10 μM CP55,940 to define non-specific binding

Both CB₁ and CB₂ membranes may be purchased from Perkin-Elmer. Dilute the concentrations so that each well received ˜8 ug protein.

Procedure:

1. Assay Set Up

20 μL CB₁ compound or buffer
180 μL radioligand
200 μL membranes
400 μL Total volume

Set up the selective CB₁ antagonist compounds in 96-well plates, with 4 compounds/plate in duplicate plates. Control samples are in the first column of the plate, and non-specific is in the last column.

2. Incubate 1-1½ hours at room temperature

3. Filter through GF/C plates soaked in 0.3% PEI. Wash with buffer plus ions and 1 mg/mL BSA.

Functional Assay for CB₁ Antagonist

Guanidine Triphosphate yS (GTPyS) Protocol
1.) Add 155 μL of membrane dilution (12.9 μg membrane/3.9 μM Guanidine Diphosphate (GDP)).
2.) Add 10 μL of 20× Inverse Agonist/Antagonist (dilute in 10% DMSO for a final concentration of 1% DMSO).
3.) Preincubate 30 minutes at room temperature.
4.) Add 10 μL of distilled H₂O, GTPyS or Agonist (dilute in 10% DMSO for a final concentration of 1% DMSO)
   a.) Add 10 μL of Vehicle only for control wells
   b.) Add 10 μL of 20× (200 μM) GTPyS to Non-Specific Binding wells.
   c.) Add 10 μL of 20× Agonist stock for stimulated wells.
5.) Incubate 60 minutes at room temperature (Soak GF/B unifilter plates in Na₂HPO₄ buffer for at least 1 hour).
6.) To start assay, add 25 μL of ³⁵S-GTPyS stock and incubate 30 minutes at room temperature (30 μL of 1 μCi/μL stock in 8.4 mL dH₂O).
   Treatment of Hypercholesterolemic/Diet Induced Obese C57BL/6 Mice with Ezetimibe

The hypercholesterolemic/diet induced obese C57BL/6 mouse can be used to evaluate the vivo efficacy of a cholesterol absorption inhibitor, ezetimibe, in combination with a selective CB₁ receptor antagonist, rimonabant. Feeding mice a “western” diet containing 45 kcal % of fat and 0.15% cholesterol diet for 21 days increased plasma cholesterol to 150 mg/dL and increased hepatic cholesteryl esters 2-fold. Ezetimibe treatment (5 mg/kg/day) reduced the plasma cholesterol levels to 102 mg/dL and completely inhibited the accumulation of hepatic cholesteryl esters with 12.8 mg/g and 4.6 mg/g in the control and ezetimibe treated mice, respectively.

Ezetimibe treatment does not appear to cause any changes in food consumption, body weights, or plasma leptin levels (van Heek, M., Austin, T. M., Farley, C., Cook, J. A., Tetzloff, G. G., Davis, H. R.: Ezetimibe, a potent cholesterol absorption inhibitor, normalizes combined dyslipidemia in obese, hyperinsulinemic hamsters. Diabetes 50:1330-1335, 2001).

Treatment of Hypercholesterolemic/Diet Induced Obese C57BL/6 Mice with Rimonabant

Diet induced obese mice (fed the “western” diet containing 45 kcal % of fat for 16 weeks) treated with the selective CB₁ receptor antagonist rimonabant once a day for 5 consecutive days at 1, 3, and 10 mg/kg p.o. showed a significant dose dependent reduction in cumulative food intake, body weight and adiposity, plasma insulin and plasma leptin levels at all doses.

Rimonabant does not appear to reduce plasma cholesterol levels (Trillou, C. R., Amone, M., Delgorge, C., Gonalons, N., Keane, P., Maffrand, J., Soubrie, P.: Anti-obesity effect of SR141716, a CB1 receptor antagonist, in diet-induced obese mice. Am J. Physiol. Regul. Integr. Comp. Physiol. 284: R345-R353, 2003).

A compound which blocks dietary cholesterol absorption would reduce the accumulation of hepatic cholesteryl esters and reduce plasma cholesterol levels, while a selective CB₁ receptor antagonist will reduce adiposity and plasma leptin and insulin levels. The combination of a cholesterol absorption inhibitor and a selective CB₁ receptor antagonist should be an effective treatment for hyperlipidemia, obesity, and metabolic syndrome.

Nonfasted plasma cholesterol levels were determined by a modification of the cholesterol oxidase method, in which the reagents were available in a kit form from Wako Pure Chemicals Industries, Ltd. (Osaka, Japan). Samples of liver (0.2 g) were lipid extracted. Lipid extracts were dried under nitrogen into HPLC sample vials, resuspended in hexane and injected onto a Zorbax Sil (4.6×25 cm) silica column. Chromatography was performed using an isocratic mobile phase containing 98.8% hexane and 1.2% isopropanol at a flow rate of 2 mL/min. Lipids were detected by absorbance at 206 nm and quantitated by computer integration (System Gold, Beckman) of elution profiles. Elution time for cholesteryl ester was 1.45 min. Cholesteryl ester content of liver-derived samples was derived from a standard curve constructed using known amounts of cholesteryl oleate. Cholesteryl oleate was used as the standard since this is the major cholesteryl ester species present in the liver and this specific cholesteryl ester has an extinction coefficient that approximates that of a weighted average for all the cholesteryl esters present in the liver.

Plasma leptin and insulin were determined using commercially available ELISA kits (Crystal Chem and ALPCO for leptin and insulin, respectively). Whole body adiposity was determined using an NMR based method (EchoMRI, Echo Medical Inc.).

It will be appreciated by those skilled in the art that changes could be made to the embodiments described above without departing from the broad inventive concept thereof. It is understood, therefore, that this invention is not limited to the particular embodiments disclosed, but it is intended to cover modifications which are within the spirit and scope of the invention, as defined by the appended claims.

Therefore, I claim:

1. A composition comprising:
(a) at least one selective CB₁ receptor antagonist; and
(b) at least one cholesterol lowering compound.

2. A composition comprising:
(a) at least one selective CB₁ receptor antagonist; and
(b) at least one sterol absorption inhibitor or at least one 5α-stanol absorption inhibitor.

3. A composition comprising:
(a) at least one selective CB₁ receptor antagonist or a pharmaceutically acceptable salt, solvate, or ester thereof; and
(b) at least one substituted azetidinone compound or substituted α-lactam compound or a pharmaceutically acceptable salt, solvate, or ester thereof.

4. A composition comprising:
(a) at least one selective CB₁ receptor antagonist or a pharmaceutically acceptable salt, solvate, or ester thereof; and
(b) at least one sterol absorption inhibitor represented by Formula (I): 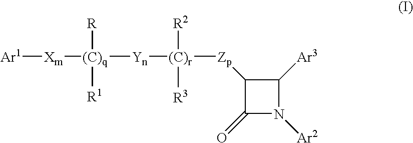
or pharmaceutically acceptable salts, solvate, or esters thereof,
wherein in Formula (I) above:
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(di-lower alkyl)-;
R and R² are independently selected from the group consisting of —OR⁶, —OC(O)R⁶, —OC(O)OR⁹ and —OC(O)NR⁶R⁷;
R¹ and R³ are independently selected from the group consisting of hydrogen, lower alkyl and aryl;
q is 0 or 1;
r is 0 or 1;
m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO2R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

5. A composition comprising:
(a) at least one selective CB1 receptor antagonist; and
(b) a compound represented by Formula (II) below: 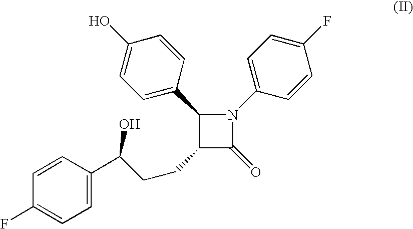
or a pharmaceutically acceptable salt, solvate, or ester thereof.

6. A therapeutic combination comprising:
(a) a first amount of at least one selective CB1 receptor antagonist; and
(b) a second amount of at least one cholesterol lowering compound or pharmaceutically acceptable salt, solvate, or ester thereof;
wherein the first amount and the second amount together comprise a therapeutically effective amount for the treatment or prevention of a vascular condition, diabetes, obesity, metabolic syndrome, or lowering a concentration of a sterol in plasma of a subject.

7. A therapeutic combination comprising:
(a) a first amount of at least one selective CB₁ receptor antagonist or a pharmaceutically acceptable salt, solvate, or ester thereof; and
(b) a second amount of at least one sterol absorption inhibitor or at least one 5α-stanol absorption inhibitor, or a pharmaceutically acceptable salt, solvate or ester thereof;
wherein the first amount and the second amount together comprise a therapeutically effective amount for the treatment or prevention of a vascular condition, diabetes, obesity, metabolic syndrome, or lowering a concentration of a sterol in plasma of a subject.

8. A therapeutic combination comprising:
(a) a first amount of at least one selective CB₁ receptor antagonist or a pharmaceutically acceptable salt, solvate, or ester thereof; and
(b) a second amount of at least one substituted azetidinone compound or substituted β-lactam compound or salt, solvate, or ester thereof;
wherein the first amount and the second amount together comprise a therapeutically effective amount for the treatment or prevention of a vascular condition, diabetes, obesity, metabolic syndrome, or lowering a concentration of a sterol in plasma of a subject.

9. A therapeutic combination comprising:
(a) a first amount of at least one selective CB₁ receptor antagonist or a pharmaceutically acceptable salt, solvate, or ester thereof; and
(b) a second amount of at least one sterol absorption inhibitor represented by Formula (I): 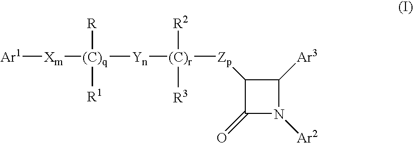
or pharmaceutically acceptable salts, solvate, or esters thereof,
wherein in Formula (I) above:
Ar¹ and Ar² are independently selected from the group consisting of aryl and R⁴-substituted aryl;
Ar³ is aryl or R⁵-substituted aryl;
X, Y and Z are independently selected from the group consisting of —CH2—, —CH(lower alkyl)- and —C(di-lower alkyl)-;
R and R² are independently selected from the group consisting of —OR⁶, —OC(O)R⁶, —OC(O)OR⁹ and —OC(O)NR⁶R⁷;
R¹ and R³ are independently selected from the group consisting of hydrogen, lower alkyl and aryl;
q is 0 or 1;
r is 0 or 1;
m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —N R⁶C(O)NR⁷R⁸, —NR⁶SO2R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl;
wherein the first amount and the second amount together comprise a therapeutically effective amount for the treatment or prevention of a vascular condition, diabetes, obesity, metabolic syndrome, or lowering a concentration of a sterol in plasma of a subject.

10. A therapeutic combination comprising:
(a) a first amount of at least one selective CB₁ receptor antagonist or a pharmaceutically acceptable salt, solvate, or ester thereof; and
(b) a second amount of a compound represented by Formula (II) below: 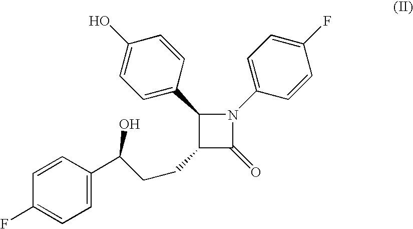
or a pharmaceutically acceptable salt, solvate, or ester thereof;
wherein the first amount and the second amount together comprise a therapeutically effective amount for the treatment or prevention of a vascular condition, diabetes, obesity, metabolic syndrome, or lowering a concentration of a sterol in plasma of a subject.

11. A pharmaceutical composition for the treatment or prevention of a vascular condition, diabetes, obesity, metabolic syndrome, or lowering a concentration of a sterol in plasma of a subject, comprising a therapeutically effective amount of a composition or therapeutic combination of claim 1 and a pharmaceutically acceptable carrier.

12. A pharmaceutical composition for the treatment or prevention of a vascular condition, diabetes, obesity, metabolic syndrome, or lowering a concentration of a sterol in plasma of a subject, comprising a therapeutically effective amount of a composition or therapeutic combination of claim 2 and a pharmaceutically acceptable carrier.

13. A pharmaceutical composition for the treatment or prevention of a vascular condition, diabetes, obesity, metabolic syndrome, or lowering a concentration of a sterol in plasma of a subject, comprising a therapeutically effective amount of a composition or therapeutic combination of claim 3 and a pharmaceutically acceptable carrier.

14. A pharmaceutical composition for the treatment or prevention of a vascular condition, diabetes, obesity, metabolic syndrome, or lowering a concentration of a sterol in plasma of a subject, comprising a therapeutically effective amount of a composition or therapeutic combination of claim 4 and a pharmaceutically acceptable carrier.

15. A pharmaceutical composition for the treatment or prevention of a vascular condition, diabetes, obesity, metabolic syndrome, or lowering a concentration of a sterol in plasma of a subject, comprising a therapeutically effective amount of a composition or therapeutic combination of claim 5 and a pharmaceutically acceptable carrier.

16. A method of treating or preventing a vascular condition, diabetes, obesity, metabolic syndrome, or lowering a concentration of a sterol in plasma of a subject, comprising the step of administering to a mammal in need of such treatment an effective amount of a composition or therapeutic combination of claim 1.

17. A method of treating or preventing a vascular condition, diabetes, obesity, metabolic syndrome, or lowering a concentration of a sterol in plasma of a subject, comprising the step of administering to a mammal in need of such treatment an effective amount of a composition or therapeutic combination of claim 2.

18. A method of treating or preventing a vascular condition, diabetes, obesity, metabolic syndrome, or lowering a concentration of a sterol in plasma of a subject, comprising the step of administering to a mammal in need of such treatment an effective amount of a composition or therapeutic combination of claim 3.

19. A method of treating or preventing a vascular condition, diabetes, obesity, metabolic syndrome, or lowering a concentration of a sterol in plasma of a subject, comprising the step of administering to a mammal in need of such treatment an effective amount of a composition or therapeutic combination of claim 4.

20. A method of treating or preventing a vascular condition, diabetes, obesity, metabolic syndrome, or lowering a concentration of a sterol in plasma of a subject, comprising the step of administering to a mammal in need of such treatment an effective amount of a composition or therapeutic combination of claim 5.

21. A composition comprising:
(a) rimonabant; and
(b) at least one cholesterol lowering compound or salt, solvate, or ester thereof.

22. A composition comprising:
(a) rimonabant; and
(b) at least one sterol absorption inhibitor or at least one 5α-stanol absorption inhibitor, or a pharmaceutically acceptable salt, solvate, or ester thereof.

23. A composition comprising:
(a) rimonabant; and
(b) at least one substituted azetidinone compound or substituted β-lactam compound or a pharmaceutically acceptable salt, solvate, or ester thereof.

24. A composition comprising:
(a) rimonabant; and
(b) at least one sterol absorption inhibitor represented by Formula (I): 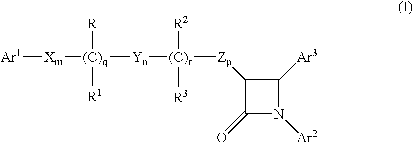
or pharmaceutically acceptable salts, solvate, or esters thereof,
wherein in Formula (I) above:
Ar¹ and Ar² are independently selected from the group consisting of aryl and R⁴-substituted aryl;
Ar³ is aryl or R⁵-substituted aryl;
X, Y and Z are independently selected from the group consisting of —CH2—, —CH(lower alkyl)- and —C(di-lower alkyl)-;
R and R² are independently selected from the group consisting of —OR⁶, —OC(O)R⁶, —OC(O)OR⁹ and —OC(O)NR⁶R⁷;
R¹ and R³ are independently selected from the group consisting of hydrogen, lower alkyl and aryl;
q is 0 or 1;
r is 0 or 1;
m, n and p are independently selected from 0, 1, 2, 3 or 4; provided that at least one of q and r is 1, and the sum of m, n, p, q and r is 1, 2, 3, 4, 5 or 6; and provided that when p is O and r is 1, the sum of m, q and n is 1, 2, 3, 4 or 5;
R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO2R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

25. A composition comprising:
(a) rimonabant; and
(b) a compound represented by Formula (II) below: 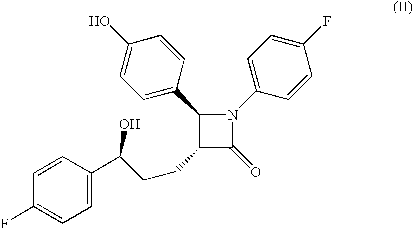
or a pharmaceutically acceptable salt, solvate, or ester thereof.

26. The method of claim 16, wherein the selective CB₁ receptor antagonist is rimonabant.

27. The method of claim 17, wherein the selective CB₁ receptor antagonist is rimonabant.

28. The method of claim 18, wherein the selective CB₁ receptor antagonist is rimonabant.

29. The method of claim 19, wherein the selective CB₁ receptor antagonist is rimonabant.

30. The method of claim 20, wherein the selective CB₁ receptor antagonist is rimonabant.

31. A method of treating or preventing a vascular condition, diabetes, obesity, metabolic syndrome, or lowering a concentration of a sterol in plasma of a subject, comprising the step of administering to a mammal in need of such treatment an effective amount of rimonabant and ezetimibe.

32. The method of claim 31, wherein said administering comprises administering rimonabant and ezetimibe in different dosage units.

33. The method of claim 32, wherein rimonabant and ezetimibe are administered simultaneously in different dosage units.

34. The method of claim 32, wherein rimonabant and ezetimibe are administered sequentially in different dosage units.

35. The method of claim 31, wherein said administering comprises administering rimonabant and ezetimibe in the same dosage unit.

36. The method of claim 31, wherein the amount of said rimonabant and the amount of said ezetimibe are the same.

37. The method of claim 31, wherein the amount of said rimonabant and the amount of said ezetimibe are different.

38. The method of claim 32, wherein the amount of said rimonabant and the amount of said ezetimibe are the same.

39. The method of claim 32, wherein the amount of said rimonabant and the amount of said ezetimibe are different.
